(19) United States
(12) Patent Application Publication
(10) Pub. No.: US 2026/0327320 A1
(43) Pub. Date: Oct. 22, 2026

(54) TREATMENT OF LUPUS

(71) Applicant: ASTRAZENECA AB, Sodertalje (SE)

(72) Inventors: Raj TUMMALA, Wilmington, DE (US); Alessandro SORRENTINO, Cambridge (GB); Heidi A. STIRNADEL-FARRANT, Cambridge (GB)

(21) Appl. No.: 19/485,479

(22) PCT Filed: May 16, 2024
(86) PCT No.: PCT/IB2024/054759

§ 371 Date: Nov. 18, 2025

Related U.S. Application Data
(60) Provisional application No. 63/503,237, filed on May 19, 2023.

(51) Int. Cl. C07K 16/28; A61K 39/00; A61P 37/02

(52) U.S. Cl. CPC C07K 16/2866; A61P 37/02; A61K 2039/505; A61K 2039/545; C07K 2317/21; C07K 2317/565; C07K 2317/71; C07K 2317/76

(57) ABSTRACT

The disclosure relates to methods and compositions for the treatment of SLE. Specifically, the disclosure relates to methods comprising administering to a subject a type I IFN receptor inhibitor.

### 1. BACKGROUND

Systemic lupus erythematosus (SLE) is a chronic, multisystemic, disabling autoimmune rheumatic disease of unknown etiology. There is substantial unmet medical need in the treatment of SLE, particularly in subjects with moderate or severe disease. Long-term prognosis remains poor for many subjects. SLE remains a disease with a highly variable course and considerable inter-individual variability [1]. There is an ongoing need for a more targeted approach to the treatment of SLE [1].

A significant problem associated with the treatment of SLE, is the heterogeneous clinical manifestations of SLE [2]. Any organ may be affected in SLE, with the skin, joints, and kidneys being the most commonly involved [3-5]. Incomplete disease control leads to progressive organ damage, poor quality of life, and increased mortality, with approximately half of all patients with SLE developing organ damage within 10 years of diagnosis [6,7]. There remains the need for a medical intervention that improves SLE disease activity across multiple systems.

The difficulty in developing effective therapeutics for SLE leads to an even higher failure rate of therapeutics in this area in clinical trials, compared to therapeutics for other indications. The development of novel therapeutics for the treatment of SLE has thus proved extremely difficult. There are many examples of clinical candidates that showed promise at Phase II but failed to show efficacy and/or safety in subsequent Phase or Phase 11 trials.

Type I interferons (IFNs) play a central role in the pathogenesis and disease course of SLE. Although these cytokines are secreted by innate immune cells to activate the immune system and defend against viral and bacterial infections, overexpression of type I IFNs occurs in patients with SLE and other autoimmune diseases, and activation of the IFN pathway is associated with increased SLE disease activity.

The present invention solves one or more of the above-mentioned problems.

### 2. SUMMARY

The present invention relates to an improved treatment SLE. The invention particularly relates to a method of treating lupus in a subject in need thereof, the method comprising administering an inhibitor of type I IFN signaling, wherein the subject was diagnosed with SLE at an age of less than (<) 50 years. The invention also relates to method of treating lupus in a subject in need thereof, the method comprising administering an inhibitor of type I IFN signaling, the subject experienced their first SLE manifestation at an age of about 30 years or less.

The invention is supported inter alia by data, presented herein for the first time, from the SLE Prospective Observation Cohort Study (SPOCS; NCT03189875). SPOCS is the first real-world study to longitudinally evaluate patients' IFNGS status. The data from SPOCS surprisingly demonstrate that a high type 1 IFN gene signature (IFNGS) is associated with an earlier SLE diagnosis and a lower age of first SLE manifestation. These insights provide opportunities for better targeting of therapies directed against the pathological type I IFN gene signature in lupus patients e.g. anifrolumab. Particularly, these data demonstrate that patients with earlier diagnosis and onset of SLE may be targeted with e.g. anifrolumab treatment based on a patient's medical history and without the need for additionally tests.

### 3. BRIEF DESCRIPTION OF FIGURES

FIG. 1: IFN scores distribution

FIG. 2: Baseline clinical manifestations by organ system involvement in the SPOCS population by IFNGS

CNS, central nervous system; IFNGS, type I interferon gene signature; SLE, systemic lupus erythematosus; SPOCS, SLE Prospective Observational Cohort Study.

FIG. 3: IFNGS status by country at baseline

The number of patients categorized as IFNGS low and IFNGS high are presented by country, with countries presented in order from smallest to largest percentage of IFNGS low patients. IFNGS, type I interferon gene signature; UK, United Kingdom; USA, United States of America.

FIG. 4: IFNGS-high and IFNGS-low patient subgroups and age of SLE diagnosis

FIG. 5: Baseline clinical manifestations by organ system involvement in the SPOCS population

FIG. 5A: Overall by baseline; FIG. 5B: SLEDAI-2K; FIG. 5C: Flares. CNS, central nervous system; SLE, systemic lupus erythematosus; SLEDAI-2K, SLE Disease Activity Index 2000; SPOCS, SLE Prospective Observational Cohort Study.

### 4. DETAILED DESCRIPTION

### 4.1. Method of Treating Lupus

The invention relates to a method of treating systemic lupus erythematous (SLE) in a subject in need thereof, the method comprising administering an inhibitor of type I IFN to the subject, wherein the method reduces SLE disease activity in the subject.

The invention relates to a method of treating SLE in a subject in need thereof, comprising administering a therapeutically effective amount of an inhibitor of type I IFN signaling, wherein a) the subject was diagnosed with SLE at an age of less than (<) 50 years, and/or b) the subject experienced their first SLE manifestation at an age of about 30 years or less. The method may comprise determining the age at which the patient was diagnosed with SLE and/or on which the subject experienced their first SLE manifestation.

The subject may have been diagnosed with SLE at an age of <30 years. The subject may have been diagnosed with SLE at an age of 18 to 29. The subject may have been diagnosed with SLE at an age of <18 years.

The inhibitor of type I IFN signaling may be a human monoclonal antibody specific for IFNAR1. The monoclonal antibody may be a modified IgG1 class human monoclonal antibody. The inhibitor of type I IFN signaling may be sifalimumab. The inhibitor of type I IFN signaling may be QX006N.

The IFNAR1 specific antibody may comprise:
(a) a heavy chain variable region complementarity determining region 1 (HCDR1) comprising the amino acid sequence of SEQ ID NO: 3;
(b) a heavy chain variable region complementarity determining region 2 (HCDR2) comprising the amino acid sequence of SEQ ID NO: 4;
c) a heavy chain variable region complementarity determining region 3 (HCDR3) comprising the amino acid sequence of SEQ ID NO: 5;
(d) a light chain variable region complementarity determining region 1 (LCDR1) comprising the amino acid sequence SEQ ID NO: 6;
(e) a light chain variable region complementarity determining region 2 (LCDR2) comprising the amino acid sequence SEQ ID NO: 7; and
(f) a light chain variable region complementarity determining region 3 (LCDR3) comprising the amino acid sequence SEQ ID NO: 8.

The IFNAR1 specific antibody may comprise: (a) a human heavy chain variable region comprising the amino acid sequence of SEQ ID NO: 1; and (b) a human light chain variable region comprising the amino acid sequence of SEQ ID NO: 2. The IFNAR1 specific antibody may comprise in the Fc region an amino acid substitution of L234F, as numbered by the EU index as set forth in Kabat and wherein said antibody exhibits reduced affinity for at least one Fc ligand compared to an unmodified antibody. The IFNAR1 specific antibody may comprise: (a) a human heavy chain comprising the amino acid sequence of SEQ ID NO: 11; and (b) a human light chain comprising the amino acid sequence of SEQ ID NO: 12.

The inhibitor of type I IFN signaling may be anifrolumab or a functional variant thereof.

The method of treatment may comprise an intravenous dose of anifrolumab or the functional variant thereof to the subject. The intravenous dose may be >300 mg anifrolumab or the functional variant thereof. The intravenous dose may be 51000 mg. The intravenous dose may be about 300 mg, about 900 mg or about 1000 mg. The intravenous dose may be administered every four weeks (Q4W)

The method of treatment may comprise administering a subcutaneous dose of anifrolumab or the functional variant thereof. The subcutaneous dose may be >105 mg and <150 mg anifrolumab or the functional variant thereof. The subcutaneous dose may be 5135 mg anifrolumab or the functional variant thereof. The subcutaneous dose may be about 120 mg. The subcutaneous dose may be administered in a single administration step. The subcutaneous dose may be administered at intervals of 6-8 days. The subcutaneous dose may be administered once per week. The subcutaneous dose may have a volume of about 0.5 to about 1 ml. The subcutaneous dose may have a volume of about 0.8 ml.

The method may comprise identifying the subject as IFNGS-test high patient pre-treatment.

The invention also relates to a pharmaceutical composition for use in the methods of the invention. The pharmaceutical composition may comprise anifrolumab or a functional variant thereof.

The invention also relates to an injection device comprising the pharmaceutical composition of the invention. The injection device may be a pre-filled syringe (PFS). The injection device may be an accessorized pre-filed syringe (AFPS). The injection device may be auto-injector.

The invention also relates to a kit comprising the injection device of the invention, and instructions for use. The instructions for use may specify that the subject a subject that was diagnosed with SLE at an age of less than (<) 50 years, and/or that a subject that experienced their first SLE manifestation at an age of about 30 years or less. The instructions for use may specify that the injection device is for use according to any of the methods of the invention.

4.2. Inhibitors of type I IFN signaling

A “type I interferon receptor inhibitor” refers to a molecule that is antagonistic for the receptor of type I interferon ligands such as interferon-α and interferon-β. Such inhibitors, subsequent to administration to a patient, may provide a reduction in the expression of at least 1 (preferably at least 4) pharmacodynamic (PD) marker genes selected from the group consisting of IFI6, RSAD2, IFI44, IFI44L, IFI27, MX1, IFIT1, HERC5, ISG15, LAMP3, OAS3, OAS1, EPST1, IFIT3, LY6E, OAS2, PLSCR1, SIGLECI, USP18, RTP4, and DNAPTP6. The at least 4 genes may suitably be IFI27, IFI44, IFI44L, and RSAD2. The “type I interferon receptor” is preferably interferon-α/β receptor (IFNAR).

For example, the type I interferon receptor inhibitor may be an antibody or antigen-binding fragment thereof that inhibits type I IFN activity (by inhibiting the receptor). An example of a suitable antibody or antigen-binding fragment thereof (that inhibits type I IFN activity) is an interferon-α/β receptor (IFNAR) antagonist. The type I interferon receptor inhibitor may be an antibody or antigen-binding fragment thereof that inhibits type I IFN activity. Additionally or alternatively, the type I interferon receptor inhibitor may be a small molecule inhibitor of a type I interferon receptor (e.g. for pharmacological inhibition of type I interferon receptor activity).

The IFNAR1 inhibitor may be a human monoclonal antibody specific for IFNAR1. The IFNAR1 inhibitor may be a modified IgG1 class human monoclonal antibody specific for IFNAR1.

The antibody may comprise a heavy chain variable region complementarity determining region 1 (HCDR1) comprising the amino acid sequence of SEQ ID NO: 3. The antibody may comprise a heavy chain variable region complementarity determining region 2 (HCDR2) comprising the amino acid sequence of SEQ ID NO: 4. The antibody may comprise a heavy chain variable region complementarity determining region 3 (HCDR3) comprising the amino acid sequence of SEQ ID NO: 5. The antibody may comprise a light chain variable region complementarity determining region 1 (LCDR1) comprising the amino acid sequence SEQ ID NO: The antibody may comprise a light chain variable region complementarity determining region 2 (LCDR2) comprising the amino acid sequence SEQ ID NO: 7. The antibody may comprise a light chain variable region complementarity determining region 3 (LCDR3) comprising the amino acid sequence SEQ ID NO: 8.

The antibody may comprise a human heavy chain variable region comprising the amino acid sequence of SEQ ID NO: 1. The antibody may comprise a human light chain variable region comprising the amino acid sequence of SEQ ID NO: 2. The antibody may comprise a human light chain constant region comprising the amino acid sequence of SEQ ID NO: 9. The antibody may comprise a human heavy chain constant region comprising the amino acid sequence of SEQ ID NO: 10. The antibody may comprise in the Fc region an amino acid substitution of L234F, as numbered by the EU index as set forth in Kabat and wherein said antibody exhibits reduced affinity for at least one Fc ligand compared to an unmodified antibody. The antibody may comprise a human heavy chain comprising the amino acid sequence of SEQ ID NO: 11. The antibody may comprise a human light chain comprising the amino acid sequence of SEQ ID NO: 12.

The antibody may comprise: (a) a heavy chain variable region complementarity determining region 1 (HCDR1) comprising the amino acid sequence of SEQ ID NO: 3; (b) a heavy chain variable region complementarity determining region 2 (HCDR2) comprising the amino acid sequence of SEQ ID NO: 4; c) a heavy chain variable region complementarity determining region 3 (HCDR3) comprising the amino acid sequence of SEQ ID NO: 5; (d) a light chain variable region complementarity determining region 1 (LCDR1) comprising the amino acid sequence SEQ ID NO: 6; (b) a light chain variable region complementarity determining region 2 (LCDR2) comprising the amino acid sequence SEQ ID NO: 7; c) a light chain variable region complementarity determining region 3 (LCDR3) comprising the amino acid sequence SEQ ID NO: 8.

The antibody may comprise (a) a human heavy chain comprising the amino acid sequence of SEQ ID NO: 11; and (b) a human light chain comprising the amino acid sequence of SEQ ID NO: 12.

The IFNAR1 inhibitor may be anifrolumab or a functional variant thereof.

### 4.3. Doses and Methods of Administration

The method may comprise administering an intravenous dose of anifrolumab or the functional variant thereof to the subject. The intravenous dose may be ≥300 mg anifrolumab or the functional variant thereof. The intravenous dose may be 51000 mg. The intravenous dose may be about 300 mg, about 900 mg or about 1000 mg. The intravenous dose may be administered every four weeks (Q4W).

The method may comprise administering a subcutaneous dose of anifrolumab or the functional variant thereof. The subcutaneous dose may be >105 mg and <150 mg anifrolumab or the functional variant thereof. The subcutaneous dose may be 5135 mg anifrolumab or the functional variant thereof. The subcutaneous dose may be about 120 mg. The subcutaneous dose may be administered in a single administration step. The subcutaneous dose may be administered at intervals of 6-8 days. The subcutaneous dose may be administered once per week. The subcutaneous dose may have a volume of about 0.5 to about 1 m. The subcutaneous dose may have a volume of about 0.8 ml.

The subject may have moderate to severe SLE pre-treatment.

The subject may be a type I interferon stimulated gene signature (IFNGS)-test high patient pre-treatment. The method may comprise identifying the subject as IFNGS-test high patient pre-treatment.

Many patients with SLE receive corticosteroids (glucocorticoids, oral corticosteroids, OCS). However, corticosteroids are associated with organ damage. Anifrolumab permits tapering of the corticosteroids (glucocorticoids) in SLE patients (steroid sparing). The method of treatment or method may comprise administering a corticosteroid to the subject, optionally wherein the corticosteroid is an oral corticosteroid. The method may comprise tapering dose of corticosteroids administered to the subject (steroid sparing). The method may comprise administering a first dose of the corticosteroid and subsequently administering a second dose of the corticosteroid, wherein the second dose of the corticosteroid is lower than the first dose of the corticosteroid. The second dose of the corticosteroid may be about a 7.5 mg prednisone-equivalent dose or less. The second dose of the corticosteroid may be a 5 mg prednisone-equivalent dose or less. The method or method of treatment may comprise administrating the second dose of the corticosteroid once per day. The first dose of the corticosteroid may be about a 10 mg prednisone-equivalent dose. The method may comprise tapering the dose of corticosteroid administered to the patient from 10 mg or more per day to less than 10 mg per day. The method or method of treatment may comprise administering the second dose of the corticosteroid once per day. The method may permit administration of a reduced dose of corticosteroids that is sustained for weeks. The second dose of the corticosteroid may be administered for at least 24 weeks. The second dose of the corticosteroid may be administered for at least 28 weeks.

The method may comprise steroid sparing in the subject, wherein the dose of the steroid administered to the subject is tapered from a pre-sparing dose at baseline to a post-sparing dose. The post-sparing dose may be 57.5 mg/day prednisone or prednisone equivalent dose. The pre-sparing dose may be 20 mg/day prednisone or prednisone equivalent dose. The steroid may comprise a glucocorticoid. The steroid may comprise an oral glucocorticoid. The steroid may be selected from the group consisting of hydrocortisone, mometasone, fluticasone, fluocinolone acetonide, fluocinolone, flurandrenolone acetonide, ciclesonide, budesonide, beclomethasone, deflazacort, flunisolide, beclomethasone dipropionate, betamethasone, betamethasone valerate, methylprednisolone, dexamethasone, prednisolone, cortisol, triamcinolone, clobetasol, clobetasol propionate, clobetasol butyrate, cortisone, corticosterone, clocortolone, dihydroxycortisone, alclometasone, amcinonide, diflucortolone valerate, flucortolone, fluprednidene, fluandrenolone, fluorometholone, halcinonide, halobetasol, desonide, diflorasone, flurandrenolide, fluocinonide, prednicarbate, desoximetasone, fluprednisolone, prednisone, azelastine, dexamethasone 21-phosphate, fludrocortisone, flumethasone, fluocinonide, halopredone, hydrocortisone 17-valerate, hydrocortisone 17-butyrate, hydrocortisone 21-acetate, prednisolone, prednisolone 21-phosphate, clobetasol propionate, triamcinolone acetonide, or a mixture thereof. The steroid may be prednisone.

The invention also relates to a unit dose for use in the methods of the invention, wherein the unit dose comprises >105 mg and ≤150 mg anifrolumab or a functional variant thereof. Unit doses of anifrolumab suitable for use in the method of the invention are described in WO2022223714 A1 [8], which is incorporated herein in its entirety.

The unit dose may comprise 5135 mg (i.e. 135 mg or less) anifrolumab or the functional variant thereof. The unit dose may comprise about 120 mg anifrolumab or the functional variant thereof. The unit dose may comprise 120 mg anifrolumab or the functional variant thereof. The unit dose may consist essentially of >105 mg and <150 mg anifrolumab or the functional variant thereof. The unit dose may consist essentially of <135 mg anifrolumab or the functional variant thereof. The unit dose may consist essentially of about 120 mg anifrolumab or the or the functional variant thereof. The concentration of anifrolumab or the functional variant thereof in the unit dose may be about 150 mg/ml. The volume of the unit dose may be less than 1 ml. The dose or unit dose may have a volume of about 0.5 to about 1 ml. The concentration of the unit dose may be about 0.8 ml. The volume of the unit dose may be 0.8 ml. The unit dose may comprise a formulation of about 150 to 200 mg/ml anifrolumab or the functional variant thereof, about 25 to 150 mM of lysine sale and an uncharged excipient. The unit dose may comprise a formulation of 150 to 200 mg/ml anifrolumab or the functional variant thereof, 25 to 150 mM of lysine sale and an uncharged excipient. The unit dose comprises a formulation of 25 mM histidine-HCL, 130 mM trehalose, and 0.05% w/v polysorbate 80. The formulation may have a pH of about 5.9.

In another aspect the invention relates to a method of treating SLE in a subject, the method comprising subcutaneously administering a dose of anifrolumab or a functional variant thereof, wherein administering the dose every week provides a plasma concentration in the subject that is at least equivalent to the plasma concentration provided by intravenous administration of 300 mg of anifrolumab or the functional variant thereof every 4 weeks. Administering the dose every week may provide a plasma concentration in the subject that is more than the plasma concentration provided by intravenous administration of 300 mg of anifrolumab or the functional variant thereof every 4 weeks. Administering the dose every week may provide a plasma concentration in the subject that is at least equivalent to the plasma concentration provided by intravenous administration of 400 mg of anifrolumab or the functional variant thereof every 4 weeks. The dose may be administered in a single-administration step. The dose administered to the subject may be <150 mg (i.e. less than 150 mg) anifrolumab or the functional variant thereof. The dose administered to the subject may be >105 mg (i.e. more than 105 mg) anifrolumab or the functional variant thereof. The dose of administered to the subject may be 5135 mg (i.e. 135 mg or less) anifrolumab or the functional variant thereof. The dose administered to the subject may be about 120 mg anifrolumab or the functional variant thereof.

Administration of the dose or unit dose may provide a plasma concentration of anifrolumab or the functional variant thereof in the patient of 10 μg (i.e. 10 μg or more) anifrolumab or the functional variant thereof per ml of plasma (i.e. a plasma concentration of 10 μg/ml). Administration of the dose or unit dose may provide a plasma concentration of anifrolumab or the functional variant thereof in the subject of about 10-100 μg/ml. Administration of the dose or unit dose may provide a plasma concentration of anifrolumab or the functional variant thereof in the subject of about 20-80 μg/ml. Administration of the dose or unit dose may provide a plasma concentration of anifrolumab or the functional variant thereof in the subject of about 30-70 μg/ml. Administration of the dose or unit dose may provide a trough concentration of anifrolumab or the functional variant thereof in the subject of 20 μg/ml (i.e. 20 μg/ml or more). Administration of the dose or unit dose may provide a trough concentration of anifrolumab or the functional variant thereof in the subject of 30 μg/ml (i.e. 30 μg/ml or more). Administration of the dose or unit dose may provide a trough concentration of anifrolumab or the functional variant thereof in the subject of 40 μg/ml (i.e. 40 μg/ml or more). Administration of the dose or unit dose may provide a trough concentration of anifrolumab or the functional variant thereof in the subject of about 20-100 μg/ml. Administration of the dose or unit dose may provide a trough concentration of anifrolumab or the functional variant thereof in the subject of about 30-80 μg/ml. Administration of the dose or unit dose may provide a trough concentration of anifrolumab or the functional variant thereof in the subject of about 40-70 μg/ml.

### 4.4. The Subject

The subject may have been diagnosed with SLE at an age of 60 or less. The subject may have been diagnosed with SLE at an age of 59 or less. The subject may have been diagnosed with SLE at an age of 58 or less. The subject may have been diagnosed with SLE at an age of 57 or less. The subject may have been diagnosed with SLE at an age of 56 or less. The subject may have been diagnosed with SLE at an age of 55 or less. The subject may have been diagnosed with SLE at an age of 54 or less. The subject may have been diagnosed with SLE at an age of 53 or less. The subject may have been diagnosed with SLE at an age of 52 or less. The subject may have been diagnosed with SLE at an age of 51 or less. The subject may have been diagnosed with SLE at an age of 50 or less. The subject may have been diagnosed with SLE at an age of 49 or less. The subject may have been diagnosed with SLE at an age of 48 or less. The subject may have been diagnosed with SLE at an age of 47 or less. The subject may have been diagnosed with SLE at an age of 46 or less. The subject may have been diagnosed with SLE at an age of 45 or less. The subject may have been diagnosed with SLE at an age of 44 or less. The subject may have been diagnosed with SLE at an age of 43 or less. The subject may have been diagnosed with SLE at an age of 42 or less. The subject may have been diagnosed with SLE at an age of 41 or less. The subject may have been diagnosed with SLE at an age of 40 or less. The subject may have been diagnosed with SLE at an age of 39 or less. The subject may have been diagnosed with SLE at an age of 38 or less. The subject may have been diagnosed with SLE at an age of 37 or less. The subject may have been diagnosed with SLE at an age of 36 or less. The subject may have been diagnosed with SLE at an age of 35 or less. The subject may have been diagnosed with SLE at an age of 34 or less. The subject may have been diagnosed with SLE at an age of 33 or less. The subject may have been diagnosed with SLE at an age of 32 or less. The subject may have been diagnosed with SLE at an age of 31 or less. The subject may have been diagnosed with SLE at an age of 30 or less.

The subject may have experienced their first SLE manifestation at an age of about 30 years or less, e.g. at an age of 30, 29, 28, 27, 26, 25, 24, 23, 22, 21 or 20. The subject may have early-onset SLE.

The subject may be a human subject. The subject may be an adult. The subject may be a patient with an elevated type I IFN gene signature. The subject may be a type I interferon stimulated gene signature (IFNGS)-test high patient pre-administration with the dose or unit dose. The subject may have elevated of the genes IFI27, IFI44, IFI44L, and RSAD2 in the whole blood. The method may comprise identifying the subject as IFNGS-test high patient pre-treatment with the dose or unit dose. The method may comprise measuring the expression of the genes IFI27, IFI44, IFI44L, and RSAD2 in the whole blood of the subject. The method may comprise measuring the expression of the genes IFI27, IFI44, IFI44L, and RSAD2 in the whole blood of the subject by RT-PCR.

The dose or unit dose may provide a therapeutic effect in the subject that is at least equivalent to a therapeutic effect provided by administration of an intravenous dose of 300 mg anifrolumab, or the functional variant thereof administered once every (Q4W). The dose or unit dose may provide a trough concentration of anifrolumab or the functional variant thereof in the subject that is greater than a trough concentration of anifrolumab or the functional variant thereof provided by administration of an intravenous dose of 300 mg anifrolumab or the functional variant thereof once every 4 weeks (Q4W). The anifrolumab or the functional variant thereof may be comprised within a pharmaceutical composition. The pharmaceutical composition may comprise about 150 to 200 mg/ml anifrolumab or the functional variant thereof, about 25 to 150 mM of lysine sale and an uncharged excipient. The pharmaceutical composition may comprise 150 mg/mL anifrolumab or the functional variant thereof. The pharmaceutical composition may comprise 50 mM lysine HCl. The pharmaceutical composition may comprise 130 mM trehalose dihydrate. The pharmaceutical composition may comprise 0.05% polysorbate 80. The pharmaceutical composition may comprise 25 mM histidine/histidine HCl. The pharmaceutical composition may comprise 150 mg/mL anifrolumab or the functional variant thereof, 50 mM lysine HCl, 130 mM trehalose dihydrate, 0.05% polysorbate 80 and 25 mM histidine/histidine HCl.

The methods of the invention may comprise administering the dose or unit dose at intervals of 6-8 days. The dose or unit dose may be administered once per week (QW). The dose or unit dose may be 120 mg anifrolumab or the functional variant thereof, wherein the method comprises administering the dose in a single administration step once per week (QW). In other words, the method comprises administering 120 mg QW of anifrolumab of the functional variant thereof. The dose or unit dose may be administered once per week for at least about 4 weeks. The dose or unit dose may be administered once per week for at least about 8 weeks. The dose or unit dose may be administered once per week for at least about 12 weeks. The dose or unit dose may be administered once per week for at least about 16 weeks. The dose or unit dose may be administered once per week for at least about 20 weeks. The dose or unit dose may be administered once per week for at least about 24 weeks. The dose or unit dose may be administered once per week for at least about 28 weeks. The dose or unit dose may be administered once per week for at least about 32 weeks. The dose or unit dose may be administered once per week for about 8 weeks. The dose or unit dose may have a volume permitted it suitable delivery in a single subcutaneous administration step. The dose or unit dose may have a volume of about 0.5 to about 1 ml. The dose or unit dose may have a volume of less than 1 ml. The dose or unit dose may have a volume of about 0.8 ml.

### 4.5. Pharmaceutical Composition

The invention also relates to a pharmaceutical composition for use in a method of treating SLE in a subject, the method comprising subcutaneously administering the pharmaceutical composition to a subject, wherein the pharmaceutical composition comprises a dose of anifrolumab or functional variant thereof, wherein the dose is >105 mg and <150 mg. The dose of anifrolumab of the functional variant thereof may be a unit dose (unit dose form, pharmaceutical unit dose form, pharmaceutical unit dose). Functional anifrolumab variants include antigen-binding fragments of anifrolumab and antibody and immunoglobulin derivatives of anifrolumab.

In another aspect the invention relates to a pharmaceutical composition for use in a method of treating CLE in a subject, the method comprising subcutaneously administering the pharmaceutical composition to the subject, wherein the pharmaceutical composition comprises a dose of anifrolumab or functional variant thereof, wherein administering the pharmaceutical composition every week provides a plasma concentration in the subject that is at least equivalent to the plasma concentration provided by intravenous administration of 300 mg of anifrolumab or the functional variant thereof every 4 weeks. Administering the dose every week may provide a plasma concentration in the subject that is about equivalent to the plasma concentration provided by intravenous administration of 400 mg of anifrolumab or the functional variant thereof every 4 weeks. The dose may be <150 mg (i.e. less than 150 mg) anifrolumab or the functional variant thereof. The dose may be >105 mg (i.e. more than 105 mg) anifrolumab or the functional variant thereof. The dose may be ≤135 mg (i.e. 135 mg or less) anifrolumab or the functional variant thereof. The dose may be about 120 mg anifrolumab or the functional variant thereof. The dose may be 120 mg anifrolumab or the functional variant thereof.

The pharmaceutical composition may be administered at intervals of 6-8 days. The pharmaceutical composition may be administered once per week (QW). The pharmaceutical composition may be administered in a single administration step. The dose may be 120 mg anifrolumab or the functional variant thereof, and the method of treatment may comprise administering the dose in a single administration step once per week (QW). The pharmaceutical composition may be administered once per week for at least about 4 weeks. The pharmaceutical composition may be administered once per week for at least about 8 weeks. The dose or unit dose may be administered once per week for at least about 12 weeks. The pharmaceutical composition may be administered once per week for at least about 16 weeks. The pharmaceutical composition may be administered once per week for at least about 20 weeks. The pharmaceutical composition may be administered once per week for at least about 24 weeks. The pharmaceutical composition may be administered once per week for at least about 28 weeks. The pharmaceutical composition may be administered once per week for at least about 32 weeks. The pharmaceutical composition may be administered once per week for about 8 weeks. The pharmaceutical composition may have a volume permitted it suitable delivery in a single subcutaneous administration step. The pharmaceutical composition may have a volume of about 0.5 to about 1 ml. The pharmaceutical composition may have a volume of less than 1 ml. The pharmaceutical composition may have a volume of about 0.8 ml.

Administration of the pharmaceutical composition may provide a plasma concentration of anifrolumab or the functional variant thereof in the patient of 10 μg (i.e. 10 μg or more) anifrolumab or the functional variant thereof per ml of plasma (i.e. a plasma concentration of 10 μg/ml). Administration of the pharmaceutical composition may provide a plasma concentration of anifrolumab or the functional variant thereof in the subject of about 10-100 μg/ml. Administration of the pharmaceutical composition may provide a plasma concentration of anifrolumab or the functional variant thereof in the subject of about 20-80 μg/ml. Administration of the pharmaceutical composition may provide a plasma concentration of anifrolumab or the functional variant thereof in the subject of about 30-70 μg/ml. Administration of the pharmaceutical composition may provide a trough concentration of anifrolumab or the functional variant thereof in the subject of 20 μg/ml (i.e. 20 μg/ml or more). Administration of the pharmaceutical composition may provide a trough concentration of anifrolumab or the functional variant thereof in the subject of 30 μg/ml (i.e. 30 μg/ml or more). Administration of the pharmaceutical composition may provide a trough concentration of anifrolumab or the functional variant thereof in the subject of 40 μg/ml (i.e. 40 μg/ml or more). Administration of the pharmaceutical composition may provide a trough concentration of anifrolumab or the functional variant thereof in the subject of about 20-100 μg/ml. Administration of the pharmaceutical composition may provide a trough concentration of anifrolumab or the functional variant thereof in the subject of about 30-80 μg/ml. Administration of the pharmaceutical composition may provide a trough concentration of anifrolumab or the functional variant thereof in the subject of about 40-70 μg/ml.

The pharmaceutical composition may provide a therapeutic effect in the subject that is at least equivalent to a therapeutic effect provided by administration of an intravenous dose of 300 mg anifrolumab or the functional variant thereof administered once every (Q4W). The pharmaceutical composition may provide a trough concentration of anifrolumab or the functional variant thereof in the subject that is greater than a trough concentration of anifrolumab or the functional variant thereof provided by administration of an intravenous dose of 300 mg anifrolumab or the functional variant thereof once every 4 weeks (Q4W). The anifrolumab or the functional variant thereof may be comprised within a pharmaceutical composition. The pharmaceutical composition may comprise about 150 to 200 mg/ml anifrolumab or the functional variant thereof, about 25 to 150 mM of lysine sale and an uncharged excipient. The pharmaceutical composition may comprise 150 mg/mL anifrolumab or the functional variant thereof. The pharmaceutical composition may comprise 50 mM lysine HCl. The pharmaceutical composition may comprise 130 mM trehalose dihydrate. The pharmaceutical composition may comprise 0.05% polysorbate 80. The pharmaceutical composition may comprise 25 mM histidine/histidine HCl. The pharmaceutical composition may comprise 150 mg/mL anifrolumab or the functional variant thereof, 50 mM lysine HCl, 130 mM trehalose dihydrate, 0.05% polysorbate 80 and 25 mM histidine/histidine HCl.

The pharmaceutical composition may comprise about 150 to 200 mg/ml anifrolumab or the functional variant thereof, about 25 to 150 mM of lysine sale and an uncharged excipient. The pharmaceutical composition may comprise 150 mg/mL anifrolumab or the functional variant thereof. The pharmaceutical composition may comprise 50 mM lysine HCl. The pharmaceutical composition may comprise 130 mM trehalose dihydrate. The pharmaceutical composition may comprise about 150 to 200 mg/ml anifrolumab or the functional variant thereof, about 25 to 150 mM of lysine sale and an uncharged excipient. The pharmaceutical composition may comprise 150 mg/mL anifrolumab or the functional variant thereof. The pharmaceutical composition may comprise 50 mM lysine HCl. The pharmaceutical composition may comprise 130 mM trehalose dihydrate. The pharmaceutical composition may comprise 0.05% polysorbate 80. The pharmaceutical composition may comprise 25 mM histidine/histidine HCl. The pharmaceutical composition may comprise 150 mg/mL anifrolumab or the functional variant thereof, 50 mM lysine HCl, 130 mM trehalose dihydrate, 0.05% polysorbate 80 and 25 mM histidine/histidine HCl.

### 4.6. Device

The invention also relates to an injection device comprising the unit dose of the invention, or the pharmaceutical composition for the use of any of the invention.

The pharmaceutical in the injection device may comprise >105 mg (i.e. more than 105 mg) and <150 mg (i.e. less than 150 mg) anifrolumab or a functional variant thereof. The pharmaceutical composition in the injection device may comprise about 120 mg anifrolumab or the functional variant thereof. The pharmaceutical composition in the injection device may comprise 120 mg anifrolumab or the functional variant thereof. The concentration of anifrolumab or the functional variant thereof in the pharmaceutical composition in the injection device may be 150 mg/ml. The volume of the pharmaceutical composition in the injection device may be at least about 0.8 ml. The volume of the pharmaceutical composition may be about 0.8 ml.

The pharmaceutical composition in the injection device may comprise about 150 to 200 mg/ml anifrolumab or the functional variant thereof, about 25 to 150 mM of lysine sale and an uncharged excipient. The pharmaceutical composition in the injection device may comprise 150 mg/mL anifrolumab or the functional variant thereof. The pharmaceutical composition in the injection device may comprise 50 mM lysine HCl. The pharmaceutical composition may comprise 130 mM trehalose dihydrate. The pharmaceutical composition in the injection device may comprise about 150 to 200 mg/ml anifrolumab or the functional variant thereof, about 25 to 150 mM of lysine sale and an uncharged excipient. The pharmaceutical composition in the injection device may comprise 150 mg/mL anifrolumab or the functional variant thereof. The pharmaceutical composition may comprise 50 mM lysine HCl. The pharmaceutical composition in the injection device may comprise 130 mM trehalose dihydrate. The pharmaceutical composition in the injection device may comprise 0.05% polysorbate 80. The pharmaceutical composition in the injection device may comprise 25 mM histidine/histidine HCl. The pharmaceutical composition in the injection device may comprise 150 mg/mL anifrolumab or the functional variant thereof, 50 mM lysine HCl, 130 mM trehalose dihydrate, 0.05% polysorbate 80 and 25 mM histidine/histidine HCl.

In another aspect the invention relates to an injection device comprising a unit dose. The unit dose may comprise >105 mg (i.e. at least 105 mg) and <150 mg (i.e. less than 150 mg) anifrolumab or a functional variant thereof. The unit dose may comprise 5135 mg (i.e. 135 mg or less) anifrolumab or the functional variant thereof. The unit dose may comprise about 120 mg anifrolumab or the functional variant thereof. The unit dose in the injection device may comprise 120 mg anifrolumab or the functional variant thereof. The unit dose in the injection device may consist essentially of >105 mg and <150 mg anifrolumab or the functional variant thereof. The unit dose in the injection device may consist essentially of ≤135 mg anifrolumab or the functional variant thereof. The unit dose in the injection device may consist essentially of about 120 mg anifrolumab or the or the functional variant thereof. The concentration of anifrolumab or the functional variant thereof in the unit dose in the injection device may be about 150 mg/ml. The volume of the unit dose in the injection device may be less than 1 ml. The unit dose in the injection device may have a volume of about 0.5 to about 1 ml. The concentration of the unit dose may be about 0.8 ml. The volume of the unit dose may be 0.8 ml. The unit dose in the injection device may comprise a formulation of about 150 to 200 mg/ml anifrolumab or the functional variant thereof, about 25 to 150 mM of lysine sale and an uncharged excipient. The unit dose in the injection device may comprise a formulation of 150 to 200 mg/ml anifrolumab or the functional variant thereof, 25 to 150 mM of lysine sale and an uncharged excipient. The unit dose comprises a formulation of 25 mM histidine-HCL, 130 mM trehalose, and 0.05% w/v polysorbate 80. The formulation may have a pH of about 5.9.

The injection device may be a pre-filled syringe (PFS). The injection device may be an accessorized pre-filed syringe (AFPS). The injection device may be an auto-injector (AI).

### 4.7. Kit

In another aspect the invention relates to a kit comprising a unit dose of the invention and instructions for use, wherein the instructions for use comprise instructions for subcutaneous administration of the unit dose to a subject.

In another aspect the invention relates to a kit comprising the pharmaceutical composition for the use of the invention, wherein the instructions for use comprise instructions for subcutaneous administration of the pharmaceutical composition to a subject.

In another aspect the invention relates to a kit comprising the injection device of any of the invention, and instructions for use, wherein the instruction for use comprise instructions for use of the injection device to subcutaneously administer the unit dose or pharmaceutical composition to the subject.

The kit of the invention may comprise packaging, wherein the packaging is adapted to hold the injection device and the instructions for use. The instructions for use may be attached to the injection device. The instruction for use may comprise instructions for administration of >105 mg and <150 mg anifrolumab or functional variant thereof. The instruction for use may comprise instructions for administration of ≤135 mg anifrolumab or the functional variant thereof. The instruction for use may comprise instructions for administration of 120 mg anifrolumab or the functional variant thereof. The instruction for use may comprise instructions for administration of 120 mg anifrolumab or the functional variant thereof every 4 weeks. The instructions for use may define the subject as having a type I IFN mediated disease. The instructions may define the subject as having SLE. The instructions may define the subject as having moderate to severe SLE. The instructions for use may be written instructions.

The instructions for use may specify that the injection device, unit dose and/or pharmaceutical composition are for use in the treatment of SLE. The instructions for use comprise instructions for administration of 120 mg anifrolumab or the functional variant thereof every week.

The instructions for use may specific that the injection device, unit dose and/or pharmaceutical composition are for use in the treatment of SLE in a subject the subject is a subject that was diagnosed with SLE at an age of less than (<) 50 years, and/or that the subject experienced their first SLE manifestation at an age of about 30 years or less.

### 4.8. Formulations

The anifrolumab or the functional variant thereof may be comprised within a pharmaceutical composition. The pharmaceutical composition may comprise about 150 to 200 mg/ml anifrolumab or the functional variant thereof, about 25 to 150 mM of lysine sale and an uncharged excipient. The pharmaceutical composition may comprise 150 mg/mL anifrolumab or the functional variant thereof. The pharmaceutical composition may comprise 50 mM lysine HCl. The pharmaceutical composition may comprise 130 mM trehalose dihydrate. The pharmaceutical composition may comprise 0.05% polysorbate 80. The pharmaceutical composition may comprise 25 mM histidine/histidine HCl. The pharmaceutical composition may comprise 150 mg/mL anifrolumab or the functional variant thereof, 50 mM lysine HCl, 130 mM trehalose dihydrate, 0.05% polysorbate 80 and 25 mM histidine/histidine HCl.

Stable formulations suitable for administration to subjects and comprising anifrolumab are described in detail in U.S. patent Ser. No. 10/125,195 B1, which is incorporated herein in its in entirety.

### 5. DEFINITIONS

### 5.1. Inhibitors of Type I IFN Signaling

### 5.1.1. Anifrolumab

Anifrolumab (MEDI-546, anifro, ANI) is a human immunoglobulin G1 kappa (IgG1κ) monoclonal antibody (mAb) directed against subunit 1 of the type I interferon receptor (IFNAR1). Anifrolumab downregulates IFNAR signaling and suppresses expression of IFN-inducible genes. Disclosures related to anifrolumab can be found in U.S. Pat. Nos. 7,662,381 and 9,988,459, which are incorporated herein by reference in their entirety. Sequence information for anifrolumab is provided in Error! Reference source not found.

TABLE 1
Anifrolumab Sequences

| | |
|---|---|
| Anifrolumab VH | EVQLVQSGAEVKKPGESLKISCKGSGYIFTNYWIAWVRQMPGKGLESMGllYPGD |
| (SEQ ID NO: 1) | SDIRYSPSFQGQVTISADKSITTAYLQWSSLKASDTAMYYCARHDIEGFDYWGRG |
| | TLVTVSS |
| Anifrolumab VL | EIVLTQSPGTLSLSPGERATLSCRASQSVSSSFFAWYQQKPGQAPRLLIYGASSR |
| (SEQ ID NO: 2) | ATGIPDRLSGSGSGTDFTLTITRLEPEDFAVYYCQQYDSSAITFGQGTRLEIK |
| HCDR1 (SEQ ID NO: 3) | NYWIA |
| HCDR2 (SEQ ID NO: 4) | IIYPGDSDIRYSPSFQG |
| HCDR3 (SEQ ID NO: 5) | HDIEGFDY |
| LCDR1 (SEQ ID NO: 6) | RASQSVSSSFFA |
| LCDR2 (SEQ ID NO: 7) | GASSRAT |
| LCDR3 (SEQ ID NO: 8) | QQYDSSAIT |
| Light chain constant region | RTVAAPSVFIFPPSDEQLKSGTASVVCLLNNFYPREAKVQWKVDNALQSGNSQES |
| (SEQ ID NO: 9) | VTEQDSKDSTYSLSSTLTLSKADYEKHKVYACEVTHQGLSSPVTKSFNRGEC |
| Heavy chain constant region | ASTKGPSVFPLAPSSKSTSGGTAALGCLVKDYFPEPVTVSWNSGALTSGVHTFPA |
| (SEQ ID NO: 10) | VLQSSGLYSLSSVVTVPSSSLGTQTYICNVNHKPSNTKVDKRVEPKSCDKTHTCP |
| | PCPAPEFEGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALPASIEKTIS |
| | KAKGQPREPQVYTLPPSREEMTKNQVSLTCLVKGFYPSDIAVEWESNGQPENNYK |
| | TTPPVLDSDGSFFLYSKLTVDKSRWQQGNVFSCSVMHEALHNHYTQKSLSLSPGK |
| Heavy chain | EVQLVQSGAEVKKPGESLKISCKGSGYIFTNYWIAWVRQMPGKGLESMGIIYPGD |
| (SEQ ID NO: 11) | SDIRYSPSFQGQVTISADKSITTAYLQWSSLKASD TAMYYCARHD |
| | IEGFDYWGRGTLVTVSSASTKGPSVFPLAPSSKSTSGGTAALGCLVKDYFPEPVT |
| | VSWNSGALTSGVHTFPAVLQSSGLYSLSSVVTVPS SSLGTQTYIC |
| | NVNHKPSNTKVDKRVEPKSCDKTHTCPPCPAPEFEGGPSVFLFPPKPKDTLMISR |
| | TPEVTCVVVDVSHEDPEVKFNWYVDGVEVHNAKTK |
| | PREEQYNSTYRVVSVLTVLHQDWLNGKEYK CKVSNKALPA SIEKTISKAK |
| | GQPREPQVYTLPPSREEMTKNQVSLTCLVKGFYPSDIAVEWESNGQPENNYKTTP |
| | PVLDSDGSFFLYSKLTVDKSRWQQGNVFSCSVMHEALHNHYTQKS LSLSPGK |
| Light chain | EIVLTQSPGTLSLSPGERATLSCRASQSVS SSFFAWYQQK PGQAPRLLIY |
| (SEQ ID NO: 12) | GASSRATGIPDRLSGSGSGT DFTLTITRLE PEDFAVYYCQ QYDSSAITFG |
| | QGTRLEIKRTVAAPSVFIFPPSDEQLKSGT ASVVCLLNNF YPREAKVQWK |
| | VDNALQSGNSQESVTEQDSKDSTYSLSSTLTLSKADYEKHKVYACEVTHQGLSSP |
| | VTKSFNRGEC |

Anifrolumab is an immunoglobulin comprising an HCDR1, HCDR2 and HCDR3 of SEQ ID NO: 3, SEQ ID NO: 4, and SEQ ID NO: 5, respectively (or functional variant thereof); and an LCDR1, LCDR2 and LCDR3 of SEQ ID NO: 6, SEQ ID NO: 7, and SEQ ID NO: 8, respectively (or functional variant thereof). Anifrolumab is an immunoglobulin comprising a VH of SEQ ID NO: 1 and a VL of SEQ ID NO: 2.

The constant region of anifrolumab has been modified such that anifrolumab exhibits reduced affinity for at least one Fc ligand compared to an unmodified antibody. Anifrolumab is a modified IgG class monoclonal antibody specific for IFNAR1 comprising in the Fc region an amino acid substitution of L234F, as numbered by the EU index as set forth in Kabat (1991, NIH Publication 91-3242, National Technical Information Service, Springfield, Va.). Anifrolumab is a modified IgG class monoclonal antibody specific for IFNAR1 comprising in the Fc region an amino acid substitution of L234F, L235E and/or P331S, as numbered by the EU index as set forth in Kabat (1991, NIH Publication 91-3242, National Technical Information Service, Springfield, Va.). Anifrolumab is an antibody comprising a light chain constant region of SEQ ID NO: 9. Anifrolumab is an antibody comprising a heavy chain constant region of SEQ ID NO: 10. Anifrolumab is an antibody comprising a light chain constant region of SEQ ID NO: 9 and a heavy chain constant region of SEQ ID NO: 10. Anifrolumab is an antibody comprising a heavy chain of SEQ ID NO: 11. Anifrolumab is an antibody comprising a light chain of SEQ ID NO: 12. Anifrolumab is an antibody comprising a heavy chain of SEQ ID NO: 11 and a light chain of SEQ ID NO: 12.

Functional variants of anifrolumab are sequence variants that perform the same function as anifrolumab. Functional variants of anifrolumab are variants that bind the same target as anifrolumab and have the same effector function as anifrolumab. Functional anifrolumab variants include antigen-binding fragments of anifrolumab and antibody and immunoglobulin derivatives of anifrolumab. Functional variants include biosimilars and interchangeable products. The terms biosimilar and interchangeable product are defined by the FDA and EMA. The term biosimilar refers to a biological product that is highly similar to an approved (e.g. FDA approved) biological product (reference product, e.g. anifrolumab) in terms of structure and has no clinically meaningful differences in terms of pharmacokinetics, safety and efficacy from the reference product. The presence of clinically meaningful differences of a biosimilar may be assessed in human pharmacokinetic (exposure) and pharmacodynamic (response) studies and an assessment of clinical immunogenicity. An interchangeable product is a biosimilar that is expected to produce the same clinical result as the reference product in any given patient.

For example, a variant of the reference (anifrolumab) antibody may comprise: a heavy chain CDR1 having at most 2 amino acid differences when compared to SEQ ID NO: 3; a heavy chain CDR2 having at most 2 amino acid differences when compared to SEQ ID NO: 4; a heavy chain CDR3 having at most 2 amino acid differences when compared to SEQ ID NO: 5; a light chain CDR1 having at most 2 amino acid differences when compared to SEQ ID NO: 6; a light chain CDR2 having at most 2 amino acid differences when compared to SEQ ID NO: 7; and a light chain CDR3 having at most 2 amino acid differences when compared to SEQ ID NO: 8; wherein the variant antibody binds to the target of anifrolumab (e.g. IFNAR) and preferably with the same affinity.

A variant of the reference (anifrolumab) antibody may comprise: a heavy chain CDR1 having at most 1 amino acid difference when compared to SEQ ID NO: 3; a heavy chain CDR2 having at most 1 amino acid difference when compared to SEQ ID NO: 4; a heavy chain CDR3 having at most 1 amino acid difference when compared to SEQ ID NO: 5; a light chain CDR1 having at most 1 amino acid differences when compared to SEQ ID NO: 6; a light chain CDR2 having at most 1 amino acid difference when compared to SEQ ID NO: 7; and a light chain CDR3 having at most 1 amino acid difference when compared to SEQ ID NO: 8; wherein the variant antibody binds to the target of anifrolumab (e.g. IFNAR) optionally with the same affinity.

A variant antibody may have at most 5, 4 or 3 amino acid differences total in the CDRs thereof when compared to a corresponding reference (anifrolumab) antibody, with the proviso that there is at most 2 (optionally at most 1) amino acid differences per CDR. A variant antibody may have at most 2 (optionally at most 1) amino acid differences total in the CDRs thereof when compared to a corresponding reference (anifrolumab) antibody, with the proviso that there is at most 2 amino acid differences per CDR. A variant antibody may have at most 2 (optionally at most 1) amino acid differences total in the CDRs thereof when compared to a corresponding reference (anifrolumab) antibody, with the proviso that there is at most 1 amino acid difference per CDR.

A variant antibody may have at most 5, 4 or 3 amino acid differences total in the framework regions thereof when compared to a corresponding reference (anifrolumab) antibody, with the proviso that there is at most 2 (optionally at most 1) amino acid differences per framework region. Optionally a variant antibody has at most 2 (optionally at most 1) amino acid differences total in the framework regions thereof when compared to a corresponding reference (anifrolumab) antibody, with the proviso that there is at most 2 amino acid differences per framework region. Optionally a variant antibody has at most 2 (optionally at most 1) amino acid differences total in the framework regions thereof when compared to a corresponding reference (anifrolumab) antibody, with the proviso that there is at most 1 amino acid difference per framework region.

A variant antibody may comprise a variable heavy chain and a variable light chain as described herein, wherein: the heavy chain has at most 14 amino acid differences (at most 2 amino acid differences in each CDR and at most 2 amino acid differences in each framework region) when compared to a heavy chain sequence herein; and the light chain has at most 14 amino acid differences (at most 2 amino acid differences in each CDR and at most 2 amino acid differences in each framework region) when compared to a light chain sequence herein; wherein the variant antibody binds to the same target antigen as the reference (anifrolumab) antibody (e.g. IFNAR) and preferably with the same affinity.

The variant heavy or light chains may be referred to as “functional equivalents” of the reference heavy or light chains. A variant antibody may comprise a variable heavy chain and a variable light chain as described herein, wherein: the heavy chain has at most 7 amino acid differences (at most 1 amino acid difference in each CDR and at most 1 amino acid difference in each framework region) when compared to a heavy chain sequence herein; and the light chain has at most 7 amino acid differences (at most 1 amino acid difference in each CDR and at most 1 amino acid difference in each framework region) when compared to a light chain sequence herein; wherein the variant antibody binds to the same target antigen as the reference (anifrolumab) antibody (e.g. IFNAR) and preferably with the same affinity.

Functional variants of anifrolumab include the antibodies described in WO 2018/023976 A1, incorporated herein by reference (Table 2).

TABLE 2
anti-IFNAR antibody sequences

| | | | |
|---|---|---|---|
| H15D10 | VH | EVQLVQSGAE VKKPGESLRISC | SEQ ID NO: 13 |
| | | KGSGYTFT NYWVAWVRQM | |
| | | PGKGLESMGI IYPGDSDTRY | |
| | | SPSFQGHVTI SADKSISTAY | |
| L8C3 | VL | DIQMTQSPSSLSASLGDRVTITCRASQNVGNYLNWYQQKP | SEQ ID NO: 14 |
| | | GKAPKLLIYRASNLASGVPSRFSGSGSGTDFTLTISSLQP | |
| | | EDFATYYCQQMEHAPPTFGQGTKVEIKR | |
| L16C11 | VL | EIVLTQSPGTLSLSPGERATLSCRASQSVIGYYLAWYQQK | SEQ ID NO: 15 |
| | | PGQAPRLLIYSVSTLASGIPDRFSGSGSGTDFTLTISRLE | |
| | | PEDFAVYYCQQYYRFPITFGQGTKVEIK | |
| H19B7 | VH | EVQLVQSGAE VKKPGESLRI SCKGSGYTFT | SEQ ID NO: 16 |
| | | NYWMAWVRQM | |
| | | PGKGLESMGI IYPSDSDTRY | |
| | | SPSFQGHVTI SADKSISTAY | |
| | | LQWSSLKASD TAMYYCARHD | |
| | | VEGYDYWGQG TLVTVSS | |

Functional variants include antibodies comprising the VH amino acid sequence SEQ ID NO: 13. Functional variants include antibodies comprising the VH amino acid sequence SEQ ID NO: 16. Functional variants include antibodies comprising the VL amino acid sequence SEQ ID NO: 14. Functional variants include antibodies comprising the VL amino acid sequence SEQ ID NO: 15. Functional variants include antibodies comprising the VL amino acid sequence SEQ ID NO: 16. Functional variants include antibodies comprising the VH sequence SEQ ID NO: 13 and VL amino acid sequence SEQ ID NO: 16. Functional variants include antibodies comprising the VH sequence SEQ ID NO: 13 and VL amino acid sequence SEQ ID NO: 15. Functional variants include antibodies comprising the VH sequence SEQ ID NO: 16 and VL amino acid sequence SEQ ID NO: 15. Functional variants include antibodies comprising the VH sequence SEQ ID NO: 16 and VL amino acid sequence SEQ ID NO: 14.

IFNAR inhibitors may be a monoclonal antibody comprising the VH amino acid sequence SEQ ID NO: 13. The anti-IFNAR antibodies may comprise the VH amino acid sequence SEQ ID NO: 16. The anti-IFNAR antibodies may comprise the VL amino acid sequence SEQ ID NO: 14. The anti-IFNAR antibodies may comprise the VL amino acid sequence SEQ ID NO: 15. The anti-IFNAR antibodies may comprise the VL amino acid sequence SEQ ID NO: 16. The anti-IFNAR antibodies may comprise the VH sequence SEQ ID NO: 13 and VL amino acid sequence SEQ ID NO: 16. The anti-IFNAR antibodies may comprise the VH sequence SEQ ID NO: 13 and VL amino acid sequence SEQ ID NO: 15. The anti-IFNAR antibodies may comprise the VH sequence SEQ ID NO: 16 and VL amino acid sequence SEQ ID NO: 15. The anti-IFNAR antibodies may comprise the VH sequence SEQ ID NO: 16 and VL amino acid sequence SEQ ID NO: 14.

### 5.1.2. QX006N

Functional variants of anifrolumab and anti-IFNAR antibodies include the QX006N antibody described in CN 11327807, incorporated herein by reference.

TABLE 3
QX006N antibody sequences

| Description | SEQ ID NO | Sequence |
|---|---|---|
| QX006N (VH) | 17 | EVQLVESGGGLVQPGGSLRLSCAASGFSLSSYYMTWVRQAPGKGLEWVSVI |
| | | NVYGGTYYASWAKGRFTISRDNSKNTLYLQMNSLRAEDTAVYYCAREDVAV |
| | | YMAIDLWGQGTLVTVSS |
| QX006N (VL) | 18 | AIQMTQSPSSLSASVGDRVTITCQASQSISNQLSWYQQKPGKAPKLLIYDA |
| | | SSLASGVPSRFSGSRSGTKFTLTISSLQPEDFATYYCLGIYGDGADDGIAF |
| | | GGGTKVEIK |
| QX006N (HCDR1) | 19 | SYYMT |
| QX006N (HCDR2) | 20 | VINVYGGTYYASWAKG |
| QX006N (HCDR3) | 21 | EDVAVYMAIDL |
| QX006N (LCDR1) | 22 | QASQSISNQLS |
| QX006N (LCDR2) | 23 | DASSLAS |
| QX006N (LCDR3) | 24 | LGIYGDGADDGIA |

IFNAR inhibitors may be a monoclonal antibody comprising the VH amino acid sequence SEQ ID NO: 17. The anti-IFNAR antibodies may comprise the VL amino acid sequence SEQ ID NO: 18.

QX006N is an immunoglobulin comprising an HCDR1, HCDR2 and HCDR3 of SEQ ID NO: 19, SEQ ID NO: 20, and SEQ ID NO: 21, respectively (or functional variant thereof); and an LCDR1, LCDR2 and LCDR3 of SEQ ID NO: 22, SEQ ID NO: 23, and SEQ ID NO: 23, respectively (or functional variant thereof). QX006N is an immunoglobulin comprising a VH amino acid sequence SEQ ID NO: 17 the VL amino acid sequence SEQ ID NO: 18.

### 5.1.3. Sifalimumab

Sifalimumab (MEDI-545) is a fully human, immunoglobulin G1 κ monoclonal antibody that binds to and neutralizes the majority of IFN-α subtypes [9]. Sifalimumab is described U.S. Pat. No. 7,741,449, which is incorporated herein by reference in its entirety. The efficacy and safety of sifalimumab were assessed in a phase IIb, randomised, double-blind, placebo-controlled study (NCT01283139) of adults with moderate to severe active systemic lupus erythematosus (SLE). 431 patients were randomised and received monthly intravenous sifalimumab (200 mg, 600 mg or 1200 mg) or placebo in addition to standard-of-care medications. The primary efficacy end point was the percentage of patients achieving an SLE responder index response at week 52. Compared with placebo, a greater percentage of patients who received sifalimumab (all dosages) met the primary end point (placebo: 45.4%; 200 mg: 58.3%; 600 mg: 56.5%; 1200 mg 59.8%).

### 5.1.4. Inhibitors of Type I IFN Signaling in the Clinic

Anifrolumab safety has been evaluated in 8 blinded or open-label intravenous (IV) and subcutaneous (SC) studies: 6 studies in patients with SLE (Study 05, Study 04, Study 1013, Study 1145, and Study 08), 1 study in patients with systemic sclerosis (SSc) (Study MI-CP180), and 1 study in healthy volunteers (Study 06) (Table 4). Of these studies, two (Studies 08 and 06) employed SC anifrolumab administration. Two studies are ongoing: 1 study in patients with SLE (Study 09) and 1 study in patients with lupus nephritis (LN) (Study 07).

TABLE 4
Clinical studies

| | IFN | | | | | |
|---|---|---|---|---|---|---|
| Phase III | signaling | | | | | |
| studies | inhibitor | Name | Subjects | Admin. | Dose | CT.gov |
| Study 05 | anifrolumab | TULIP II | SLE patients | IV | 300 mg | NCT02446899 |
| | | | | | Q4W | |
| Study 04 | anifrolumab | TULIP I | SLE patients | IV | 300 mg | NCT02962960 |
| | | | | | Q4W | |
| Study 09 | anifrolumab | Long-term | SLE patients | IV | 300 mg | |
| | | extension | | | | |

Phase II studies

| | | | | | | |
|---|---|---|---|---|---|---|
| Study 1013 | anifrolumab | MUSE | SLE patients | IV | 300 mg or | NCT01438489 |
| | | | | | 1000 mg | |
| | | | | | Q4W | |
| Study 1145 | anifrolumab | MUSE | | IV | 300 mg | NCT01753193 |
| | | OLE | | | | |
| Study 08 | anifrolumab | | SLE patients | SC | | NCT02962960 |
| Study 07 | anifrolumab | | LN patients | | | NCT02547922 |
| | sifalimumab | | SLE patients | IV | 200 mg, | NCT01283139 |
| | | | | | 600 mg, or | |
| | | | | | 1200 mg | |

Phase I

| | | | | | |
|---|---|---|---|---|---|
| Study MI- | anifrolumab | Scleroderma | IV | 0.1, 0.3, | NCT00930683 |
| CP180 | | patients | | 1.0, 3.0, | |
| | | | | 5.0, 10, | |
| | | | | 20.0 mg/kg | |
| Study 06 [10] | anifrolumab | Healthy | IV and | 300 mg, | NCT02601625 |
| | | volunteers | SC | SC, 300 | |
| | | | | mg IV or | |
| | | | | 600 mg SC | |
| Study MI- | sifalimumab | Myositis | IV | 0.3, 1.0, | NCT00533091 |
| CP151 | | patients | | 3.0 or 10.0 | |
| | | | | mg/kg | |

Study MI-CP151 is described in further detail in Higgs et al. 2013 [11]. Study 1013 is described in further detail in Furie et al. 2017 [12], which is incorporated herein by reference in its entirety. Study 04 is described in further detail in Furie et al. 2019 [13], which is incorporated herein by reference in its entirety. The results of Study 05 are presented in Morand et al. 2020 [14], herein incorporated by reference in its entirety. A full summary of the evidence for intravenous anifrolumab clinical efficacy in SLE is provided in Tanaka et al., 2020 [15], which is incorporated herein by reference in its entirety.

### 5.2. Formulations

Stable formulations suitable for administration to subjects and comprising anifrolumab are described in detail in U.S. patent Ser. No. 10/125,195 B1, which is incorporated herein in its in entirety.

The Examples that follow are illustrative of specific embodiments of the disclosure, and various uses thereof. They are set forth for explanatory purposes only and should not be construed as limiting the scope of the disclosure in any way.

### 5.3. Steroids

Oral corticosteroids (OCS, glucocorticoids) include prednisone, cortisone, hydrocortisone, methylprednisolone, prednisolone and triamcinolone. Examples of equivalent doses of oral prednisone are shown in Table 5.

TABLE 5
Examples of equivalent doses of oral prednisone

| | Oral Prednisone and Equivalents | | | | |
|---|---|---|---|---|---|
| | Equivalent Dose | | | | |
| Oral Prednisone | 7.5 mg | 10 mg | 20 mg | 30 mg | 40 mg |
| Cortisone | 37.5 mg | 50 mg | 100 mg | 150 mg | 200 mg |
| Hydrocortisone | 30 mg | 40 mg | 80 mg | 120 mg | 160 mg |
| Methylprednisolone | 6 mg | 8 mg | 16 mg | 24 mg | 32 mg |
| Prednisolone | 7.5 mg | 10 mg | 20 mg | 30 mg | 40 mg |
| Triamcinolone | 6 mg | 8 mg | 16 mg | 24 mg | 32 mg |

### 5.4. Dosage Forms

A unit dose (also referred to as a unit dose form, a pharmaceutical unit dose or a pharmaceutical unit dose form) is a dose formed from a single unit. A unit dose (unit dose form) is suitable for administration to a subject in a single administration step. A unit dose (unit dose form) may be packaged in a single-unit container, for example a single-use pre-filled syringe or autoinjector. Unit doses provide the advantage that they can be ordered, packaged, handled and administered as single dose units containing a pre-determined amount of a drug. Unit doses decrease administration errors and reduce waste. Unit dose of anifrolumab suitable for use in the treatment of SLE are described in WO2022223714 A1 [8].

### 5.5. Delivery Device

As well as providing for subcutaneous administration of the antibody, the ability to self-administer (e.g. for home use) may further be enhanced by subcutaneous administration via an accessorized pre-filled syringe (APFS), an autoinjector (AI), or a combination thereof. Such devices have been found to be well-tolerated and reliable for administering subcutaneous doses of an antibody and provide further options for optimizing patient care. Indeed, such devices may reduce the burden of frequent clinic visits for patients. An example of a suitable APFS device is described in Ferguson et. al. [16], which is incorporated herein by reference in its entirety.

The dose elucidated by the inventors provides yet advantages in the context of APFS-administration, as an APFS device typically administers a maximal volume of 1 ml. A dose in the range of >105 mg to <155 mg can be readily accommodated by a volume of ~0.8 ml, such that the dose(s) of the present invention are uniquely suited to APFS and AI administration. For comparison, due to viscosity of the anifrolumab, larger doses (particularly doses of >150 mg) would need to be administered within a volume of >1 ml, requiring at least two SC injections, which is inconvenient for the patient, and would require a plurality of pre-filled devices.

The delivery device may be single use, disposable system that is designed to enable manual, SC administration of the dose.

### 5.6. End Points

### 5.6.1. Patient Reported Outcomes

Physician Global Assessment (PGA and MDGA) of Disease Activity refers to an assessment wherein a physician evaluates the status of a subject's psoriatic arthritis (PsA) by means of a visual analog scale (VAS). The subject is assessed according to how their current arthritis is. The VAS is anchored with verbal descriptors of “very good” to “very poor.”

### 5.6.2. BILAG-2004 (British Isles Lupus Assessment Group-2004)

The BILAG-2004 is a translational index with 9 organ systems (General, Mucocutaneous, Neuropsychiatric, Musculoskeletal, Cardiorespiratory, Gastrointestinal, Ophthalmic, Renal and Haematology) that is able to capture changing severity of clinical manifestations. It has ordinal scales by design and does not have a global score; rather it records disease activity across the different organ systems at a glance by comparing the immediate past 4 weeks to the 4 weeks preceding them. It is based on the principle of physicians' intention to treat and categorizes disease activity into 5 different levels from A to E:
Grade A represents very active disease requiring immunosuppressive drugs and/or a prednisone dose of >20 mg/day or equivalent
Grade B represents moderate disease activity requiring a lower dose of corticosteroids, topical steroids, topical immunosuppressives, antimalarials, or NSAIDs
Grade C indicates mild stable disease
Grade D implies no disease activity but the system has previously been affected
Grade E indicates no current or previous disease activity

Although the BILAG-2004 was developed based on the principle of intention to treat, the treatment has no bearing on the scoring index. Only the presence of active manifestations influences the scoring.

BILAG-defined improvement in mucocutaneous or musculoskeletal organ systems were representative of rash or arthritis, respectively.

### 5.6.3. BICLA (BILAG-Based Composite Lupus Assessment)

BICLA is a composite index that was originally derived by expert consensus of disease activity indices. BICLA response is defined as (1) at least one gradation of improvement in baseline BILAG scores in all body systems with moderate or severe disease activity at entry (e.g., all A (severe disease) scores falling to B (moderate), C (mild), or D (no activity) and all B scores falling to C or D); (2) no new BILAG A or more than one new BILAG B scores; (3) no worsening of total SLEDAI score from baseline; (4) no significant deterioration (≤10%) in physicians global assessment; and (5) no treatment failure (initiation of non-protocol treatment).

Particularly, a subject is a BICLA responder if the following criteria are met:
a) Reduction of all baseline BILAG-2004 A to B/C/D and baseline BILAG-2004 B to C/D, and no BILAG-2004 worsening in other organ systems, as defined by 1 new BILAG-2004 A or more than 1 new BILAG-2004 B item;
b) No worsening from baseline in SLEDAI-2K as defined as an increase from baseline of >0 points in SLEDAI-2K;
c) No worsening from baseline in the subjects' lupus disease activity defined by an increase 20.30 points on a 3-point PGA VAS;
d) No discontinuation of investigational product or use of restricted medications beyond the protocol-allowed threshold before assessment

### 5.6.4. SRI (Systemic Lupus Erythematosus Responder Index of 4)

A subject achieves SRI(4) if all of the following criteria are met:
Reduction from baseline of >4 points in the SLEDAI-2K;
No new organ system affected as defined by 1 or more BILAG-2004 A or 2 or more
BILAG-2004 B items compared to baseline using BILAG-2004;
No worsening from baseline in the subjects' lupus disease activity defined by an increase 20.30 points on a 3-point PGA VAS.

SRI(X) (X=5, 6, 7, or 8) is defined by the proportion of subjects who meet the following criteria:
Reduction from baseline of X points in the SLEDAI-2K;
No new organ systems affected as defined by 1 or more BILAG-2004 A or 2 or
more BILAG-2004 B items compared to baseline using BILAG-2004;
No worsening from baseline in the subjects' lupus disease activity defined by an
increase 20.30 points on a 3-point PGA VAS

### 5.6.5. SLEDAI-2K (Systemic Lupus Erythematosus Disease Activity Index 2000)

The SLEDAI-2K disease activity index consists of a list of organ manifestations, each with a definition. A certified Investigator or designated physician will complete the SLEDAI-2K assessment and decide whether each manifestation is “present” or “absent” in the last 4 weeks. The assessment also includes the collection of blood and urine for assessment of the laboratory categories of the SLEDAI-2K.

The SLEDAI-2K assessment consists of 24 lupus-related items. It is a weighted instrument, in which descriptors are multiplied by a particular organ's “weight”. For example, renal descriptors are multiplied by 4 and central nervous descriptors by 8 and these weighted organ manifestations are totaled into the final score. The SLEDAI-2K score range is 0 to 105 points with 0 indicating inactive disease. The SLEDAI-2K scores are valid, reliable, and sensitive clinical assessments of lupus disease activity. The SLEDAI-2K calculated using a timeframe of 30 days prior to a visit for clinical and laboratory values has been shown to be similar to the SLEDAI-2K with a 10-day window [17].

SLEDAI-2K-defined resolution of rash is defined as a score of 0 at Week 52 for those with a score >2 for rash at baseline.

### 5.7. Systemic Lupus Erythematous

Multiple lines of evidence indicate a role of type I interferons (IFNs) in the pathogenesis of SLE:
Genetic polymorphisms associated with type I IFNs are associated with susceptibility to SLE [18-20].
High IFN-α levels and type I IFN activity have been reported in SLE [21,22].
Increased levels of messenger ribonucleic acid (mRNA), whose transcription is induced by type I IFNs (type I IFN signature), are prominent in peripheral blood mononuclear cells and whole blood in approximately 60% of SLE patients and are associated with greater disease activity [23-28].
Transcripts induced by type I IFN are the most overexpressed transcripts in SLE [29].
Proteins induced by IFN are increased in patients with SLE [30-32].
Overexpression of type I IFN, type I IFN signature, and proteins induced by type I IFNs have been associated with greater disease activity and organ system involvement in SLE.

Patients with high anti-double stranded deoxyribonucleic acid (anti-dsDNA) antibody titers, lupus nephritis, and progressive skin rashes have high serum levels of type I IFN [21]. In addition, patients with acute skin involvement tend to have elevated IFN in blood and skin [22]. Skin biopsies from patients with SLE also show increased type I IFN signature [29]. Proteins induced by IFN are increased in patients with active central nervous system (CNS) symptoms [32].

Immune complexes containing SLE autoantibodies, such as anti-dsDNA or anti-ribonucleoprotein (anti-RNP) antibodies, can activate type I IFN production [21]. After internalization through Fc receptors, autoantibody-containing immune complexes bind endosomal toll-like receptor 7 and toll-like receptor 9, stimulating production of type I IFN. Type I IFN stimulates monocyte derived dendritic cell maturation, which promotes loss of tolerance and generation of autoreactive T and B cells, autoantibody production, immune complex formation, and further production of type I IFN, creating a self-perpetuating cycle of autoimmunity.

Anifrolumab is a human immunoglobulin G1 kappa (IgG1K) monoclonal antibody (mAb) directed against Type I interferon receptor (IFNAR1). Anifrolumab inhibits binding of type I IFN to IFNAR and inhibits the biologic activity of all type I IFNs. Two phase 3 studies (TULIP-1 and TULIP-2) and a phase 2b study (MUSE) have provided substantial evidence for the efficacy and safety of anifrolumab for moderately to severely active SLE. In all three studies, treatment with anifrolumab was associated with treatment differences >16% compared with placebo at Week 52 in British Isles Lupus Assessment Group-based Composite Lupus Assessment response rates. The combined data across a range of other clinically significant endpoints (e.g. oral corticosteroid reduction, improved skin disease, flare reduction) further support the efficacy of anifrolumab for SLE treatment [33].

### 5.8. Type I IFN Gene Signature (IFNGS)

Type I IFN is considered to play a central role SLE disease pathogenesis and inhibition of this pathway is targeted by anifrolumab. To understand the relationship between type I IFN expression and response to anti-IFN therapy, it is necessary to know if a subject's disease is driven by type I IFN activation. However, direct measurement of type I IFN remains a challenge. As such, a transcript-based marker was developed to evaluate the effect of over expression of the target protein on a specific set of mRNA markers. The expression of these markers is easily detected in whole blood and demonstrates a correlation with expression in diseased tissue such as skin in SLE. The bimodal distribution of the transcript scores for SLE subjects supports defining an IFN test high and low subpopulation. The type I IFN test is described in WO2011028933 A1, which is incorporated herein by reference in its entirety. The type I IFN gene signature may be used to identify a subject has a type I IFN gene signature (IFNGS)-test high patient or an IFNGS-test low patient. The IFNGS test measures expression of the genes IFI27, IFI44, IFI44L, and RSAD2 compared with 3 reference genes; 18S, ACTB and GAPDH in the whole blood of the subject. The result of the test is a score that is compared with a pre-established cut-off that classifies patients into 2 groups with low or high levels of IFN inducible gene expression.

The expression of the genes may be measured by RT-PCR. Suitable primers and probes for detection of the genes may be found in WO2011028933. A suitable kit for measuring gene expression for the IFNGS test is the QIAGEN Therascreen® IFIGx RGQ RT-PCR kit (IFIGx kit), as described in Brohawn et al. [34], which is incorporated herein by reference in its entirety. The type I IFN gene signature test measures the mRNA expression of 4 type I IFN-inducible genes (IFI27, IFI44, IFI44L, and RSAD2) relative to 3 housekeeping genes (ACTB, 18S, and GAPDH).

The type I IFNGS test may be conducted at a designated central laboratory using the QIAGEN therascreen IFN-inducible gene expression (IFIGx) Rotor-Gene Q (RGQ) reverse transcriptase PCR (RT-PCR) system. This expression system consists of collection tubes and

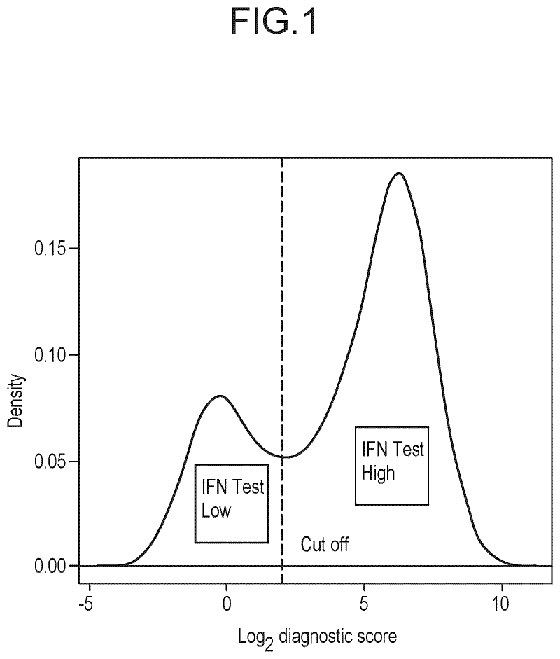

RNA isolation kits described above, along with a therascreen IFIGx RGQ RT-PCR kit used with RGQ molecular diagnostic platform with IFIGx software. The IFIGx kit measures the expression of four type I IFN-inducible genes (IFI27, IFI44, IFI44L and RSAD2) relative to three housekeeping genes (ACTB, 18S and GAPDH) and generates a qualitative diagnostic score of positive or negative. The result is expressed as a score that is compared with a pre-established delta Ct-based cut-off, in the trough of the bimodal distribution, and classifies patients into one of two groups representing low or high IFIGx [35](FIG. 1).

The analytical validation of the four-gene test as a measurement of IFIGx has been reported and was in two phase 11 studies of anifrolumab for the treatment of SLE (NCT02446899 and NCT02446912) [36]. Patients with SLE, who present heterogeneous disease activity and symptoms, are commonly stratified by low or high levels of IFIGx [37,38].

### 6. EXAMPLE: BURDEN OF SYSTEMIC LUPUS ERYTHEMATOSUS IN CLINICAL PRACTICE: BASELINE DATA FROM THE SLE PROSPECTIVE OBSERVATIONAL COHORT STUDY (SPOCS) BY INTERFERON

### 6.1. Introduction

SLE is a very heterogeneous disease. A better understanding of the differences between patients may reveal insights into disease pathogenesis, allow for better targeted therapy and will ultimately lead to improved clinical outcomes.

The SLE Prospective Observation Cohort Study (SPOCS; NCT03189875) was designed to systematically describe the patient journey in terms of clinical features, disease activity and progression, treatment patterns, clinical outcomes, patient-reported health outcomes (PROs), and healthcare resource utilization (HCRU) in patients with moderate to severe systemic lupus erythematosus (SLE), according to IFNGS status [35]. SPOCS is the first real-world study to longitudinally evaluate patients' IFNGS status. Type I IFN-inducible (IFI) gene expression, measured using the IFN Gene Signature (IFNGS), provides a method to assess type I IFN pathway activation in individual patients.

### 6.2. Methods

### 6.2.1. Study Design and Patients

SPOCS (NCT03189875) is an international, multicenter, prospective, observational cohort of patients with moderate to severe SLE. Patients were enrolled from June 2017 through December 2019 and followed for a maximum of 3 years with planned biannual study visits ending in November 2022. All treatment was based on the patient's and treating physician's decision only and not influenced by the SPOCS protocol. Demographic, clinical, and laboratory data were collected from patients enrolled in 8 countries in North America (Canada and United States of America), Europe (France, Germany, Italy, Spain, the United Kingdom), and Australia. The study was designed to be broad in enrollment. To be included in the study, patients must have been >18 years of age and provided written informed consent. All patients met American College of Rheumatology (ACR) or Systemic Lupus International Collaborating Clinics (SLICC) SLE classification criteria, had current or previous serology of antinuclear antibodies (ANA) or anti-double-stranded DNA antibodies, received a minimum of 6 months of systemic treatment (beyond non-steroidal anti-inflammatory drugs [NSAIDs] and analgesics) for active SLE, and were classified as having moderate to severe SLE (modified Systemic Lupus Erythematosus Disease Activity Index 2000 [modified SLEDAI-2K, excluding urine or laboratory results including immunologic measures and lupus headache] score >4 or SLEDAI-2K score >6 points). Patients were excluded if they had active severe lupus nephritis, were enrolled in interventional trials involving investigational agents, or were unable to complete study measures. The planned sample size of 1500 patients was amended to 900 due to slow accrual of eligible patients; accordingly, using an event of interest incidence rate of 1 event per 100 person-years in a 2-year follow-up in 25% of patients, endpoint precision estimates for the incidence rate were adjusted to 2.1 (95% confidence interval: 0.30-2.44).

SPOCS is being conducted in accordance with the principles of the Declaration of Helsinki and is consistent with the International Council for Harmonization Good Clinical Practices, Good Pharmacoepidemiology Practice, and applicable legislation according to the study classification in each country. All patients enrolled in SPOCS provided written informed consent.

### 6.2.2. Data Collection

To understand the baseline characteristics and identify any differences in the cohorts, data were collected at study entry, defined as the routine visit at the time consent was signed. Baseline data included patient demographics (e.g., age, sex, and race [American Indian or Alaska Native, Asian, Black or African American, Indigenous Australian, Native Hawaiian or Other Pacific Islander, White, or Other], or ethnicity [Hispanic or Latino, not Hispanic or Latino] as permitted by local regulations), body mass index, SLE disease characteristics including date of first diagnosis, SLEDAI-2K and modified SLEDAI-2K scores, organ system involvement and organ damage according to SLICC/ACR damage index (SDI) score, number and severity of SLE flares (mild/moderate or severe) according to Safety of Estrogens in Lupus National Assessment (SELENA)-SLEDAI Flare Index (SFI), Physician Global Assessment of Disease Activity (PGA), comorbidities, along with SLE treatments and concomitant medications. Blood samples were collected to determine baseline IFNGS.

### 6.2.3. IFNGS Testing

A blood sample was collected in a PAXgene™ tube and sent to a central laboratory for processing. Each patient's baseline mRNA expression of 4 type I IFN-inducible genes (IFI27, IFI44, IFI44L, and RSAD2) relative to 3 housekeeping genes (ACTB, 18S, and GAPDH) was determined using the QIAGEN Therascreen® IFN-IFI gene expression Rotor-Gene®Q reverse transcriptase PCR System. This expression score was compared with a pre-established cut-off to classify each patient as IFNGS high or low [29,39].

### Statistical Analysis

Demographics, clinical characteristics, and SLE medications were summarized by overall SPOCS population, SLEDAI-2K categories (<10 vs >10) [18], IFNGS status (high vs low), and countries. Continuous variables were reported as arithmetic means and standard deviation, median, first and third quartiles, and range, as appropriate. Categorical variables were summarized as number and percentages of patients with non-missing values in each category. Patient subgroups were compared using Mann-Whitney U test, chi-square test, Kruskal-Wallis test, or Fisher's exact test. All P-values presented are nominal. All calculations and analyses were performed in R version 4.1.0 (R Foundation for Statistical Computing, Vienna, Austria).

### 6.2.4. Patient and Public Involvement

No patients or members of the public were involved in the design of this trial.

### 6.3. Results

### 6.3.1. Baseline Patient Characteristics and Demographics

Between June 2017 and December 2019, 1050 patients were screened; 823 patients were enrolled from North America (n=389) and Europe or Australia (n=434). Most enrolled patients [93.2%, 767/823) were female, and the mean (SD) age at study entry was 45.3 (13.9) years (Table 6). Overall, 9.1% (71/779) of enrolled patients were Asian, 16.4% (128/779) were Black, 69.5% (541/779) were White, and 5.0% (39/779) of patients identified with a different race. The mean (SD) years since SLE diagnosis was 11.1 (9.2) years (Table 7).

When stratified by baseline disease activity scores, 241 patients had SLEDAI-2K≥10 and 343 had SLEDAI-2K<10. Patients with SLEDAI-2K>10 vs SLEDAI-2K<10 were younger (mean [SD]: 42.8 [13.7] years vs 46.6 [14.2] years; P=0.001) (Table 6). No difference in mean age at SLE first manifestation was observed between patients with SLEDAI-2K≥10 vs<10. Race distribution was similar in patients with SLEDAI-2K 210 vs SLEDAI-2K<10 (White: 69.4% (159/229) vs 67.6% (217/321), Black: 17.0% (39/229) vs 16.5% (53/321), and Asian: 8.7% (20/229) vs 10.6% (34/321); P=0.89). At baseline, the mean [SD] duration of SLE was shorter in patients with SLEDAI-2K>10 vs SLEDAI-2K<10 (10.4 [8.6] years vs 12.4 [9.6] years; P=0.012) (Table 7).

TABLE 6
Baseline demographics and patient characteristics of the SPOCS population by SLEDAI-
2K and IFNGS category

| | | SLEDAI-2K | | | IFNGS | | |
|---|---|---|---|---|---|---|---|
| | Overall | <10 | ≥10 | P- | High | Low | Nominal |
| | (N = 823) | (n = 343) | ( n = 241) | value | (n = 522) | (n = 217) | P-value |
| Age, years | n = 823 | n = 343 | n = 241 | 0.001 | n = 522 | n = 217 | <0.001 |
| Mean # SD | 45.3 ± 13.9 | 46.6 ± 14.2 | 42.8 ± 13.7 | | 43.1 ± 13.6 | 50.7 ± 12.9 | |
| Median (IQR) | 45.0 (34.0, | 47.0 (36.0, | 41.0 (33.0, | | 42.0 (32.0, | 51.0 (42.0, | |
| | 55.0) | 57.0) | 51.0) | | 54.0) | 59.0) | |
| Age >40 years, | 503 (61.1) | 229 (66.8) | 121 (50.2) | <0.001 | 283 (54.2) | 168 (77.4) | <0.001 |
| n (%) | | | | | | | |
| Male, n (%) | 56 (6.8) | 28 (8.1) | 14 (5.8) | 0.278 | 33 (6.3) | 15 (6.9) | 0.767 |
| Race, n (%) | n = 779 | n = 321 | n = 229 | 0.892 | n = 483 | n = 213 | <0.001 |
| Asian | 71 (9.1) | 34 (10.6) | 20 (8.7) | | 50 (10.4) | 10 (4.7) | |
| Black | 128 (16.4) | 53 (16.5) | 39 (17.0) | | 96 (19.9) | 15 (7.0) | |
| White | 541 (69.5) | 217 (67.6) | 159 (69.4) | | 311 (64.4) | 179 (84.0) | |
| Other | 39 (5.0) | 17 (5.3) | 11 (4.8) | | 26 (5.4) | 9 (4.2) | |
| Ethnicity, n (%) | n = 722 | n = 279 | n = 217 | | n = 449 | n = 196 | |
| Hispanic or | 113 (15.7) | 49 (17.6) | 36 (16.6) | 0.776 | 87 (19.4) | 20 (10.2) | 0.004 |
| Latino | | | | | | | |
| BMI kg/m² | n = 727 | n = 297 | n = 221 | 0.017 | n = 458 | n = 195 | <0.001 |
| Mean ± SD | 27.1 ± 6.8 | 27.5 ± 7.1 | 26.1 6.5 | | 26.6 ± 6.5 | 29.0 ± 7.3 | |
| Median (IQR) | 26.1 (22.0, | 26.3 (22.3, | 24.9 (21.0, | | 25.4 (21.8, | 27.8 (23.5, | |
| | 30.8) | 31.1) | 29.3) | | 29.8) | 32.7) | |
| BMI category, n | n = 727 | n = 298 | n = 221 | 0.190 | n = 458 | n = 195 | 0.005 |
| (%) | | | | | | | |
| Underweight | 33 (4.5) | 16 (5.4) | 15 (6.8) | | 25 (5.5) | 4 (2.1) | |
| Normal | 284 (39.1) | 106 (35.7) | 96 (43.4) | | 186 (40.6) | 63 (32.3) | |
| Overweight | 212 (29.2) | 88 (29.6) | 60 (27.2) | | 136 (29.7) | 58 (29.7) | |
| Obese | 198 (27.2) | 87 (29.3) | 50 (22.6) | | 111 (24.2) | 70 (35.9) | |

All P-values are nominal; there have been no adjustments for multiplicity. Subgroups do not total overall population n due to missing data.
BMI, body mass index;
SD, standard deviation;
IFNGS, type I interferon gene signature;
IQR, interquartile range;
SLE, systemic lupus erythematosus;
SLEDAI-2K, SLE Disease Activity Index 2000;
SPOCS, SLE Prospective Observational Cohort Study.

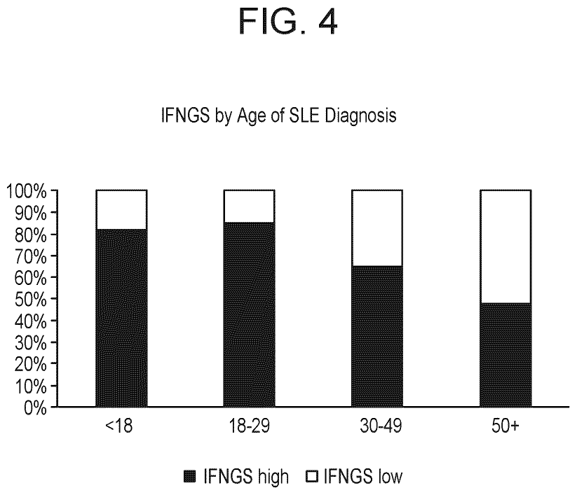

Approximately two thirds of patients were categorized as IFNGS high, while one third was IFNGS low (70.6% [522/739] vs 29.4% [217/739]) (Table 6). The proportion of patients in each IFNGS category differed across races (P<0.001); the percentages of Asian, Black, and White patients within the IFNGS-high group were 10.4%, 19.9%, and 64.4%, respectively, whereas the percentages in the IFNGS-low group were 4.7%, 7.0%, and 84.0%, respectively. Within the Asian group, 83.3% (50/60) of patients were IFNGS high and 16.7% (10/60) were IFNGS low; within the Black group, 86.5% (96/111) of patients were IFNGS high and 13.5% (15/111) were IFNGS low; and within the White group, 63.5% (311/490) of patients were IFNGS high and 36.5% (179/490) were IFNGS low. At baseline, the IFNGS-high group was younger (mean [SD] age: 43.1 [13.6] years vs 50.7 [12.9] years; P<0.001) (Table 6). A greater percentage of patients in the IFNGS-high group were <30 years of age at SLE diagnosis compared with the IFNGS-low group (50.5% vs 22.7%) (Table 7). Patients in the IFNGS-high group were younger at SLE first manifestation than those in the IFNGS-low group (mean [SD]: 30.0 [12.7] years vs 36.8 [14.6] years; P<0.001) (Table 7), with 53.7% of patients <30 years of age at first manifestation, compared with 32.3% of the IFNGS-low group (FIG. 4). Surprisingly, whilst about 50% of patients diagnosed at the age of 50+ were IFNGS-low, less than 20% of patients diagnosed at the age of 29 or less were IFNGS-low (FIG. 4).

### 6.3.2. Baseline SLE Disease Activity and Damage

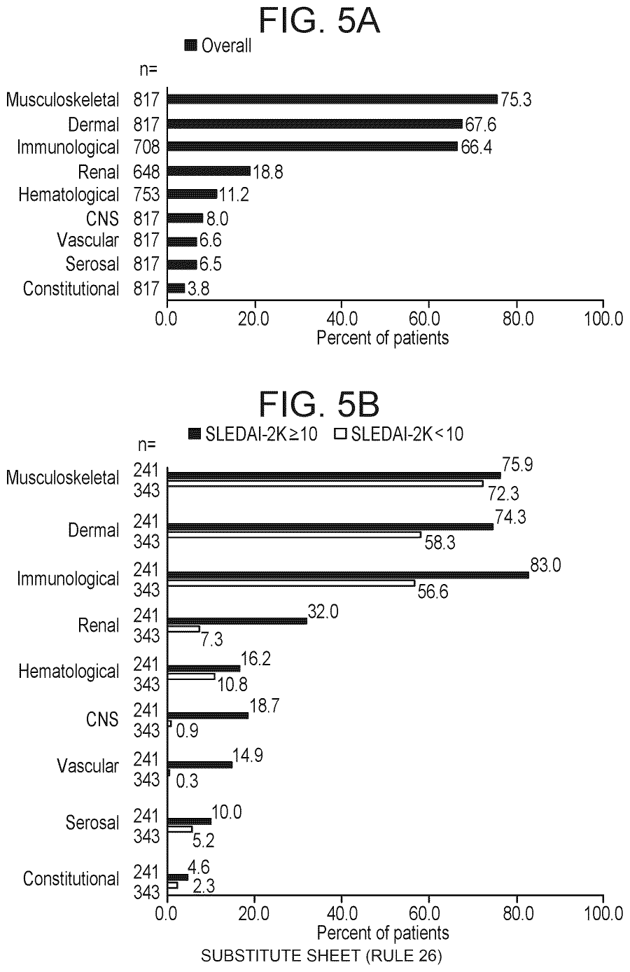

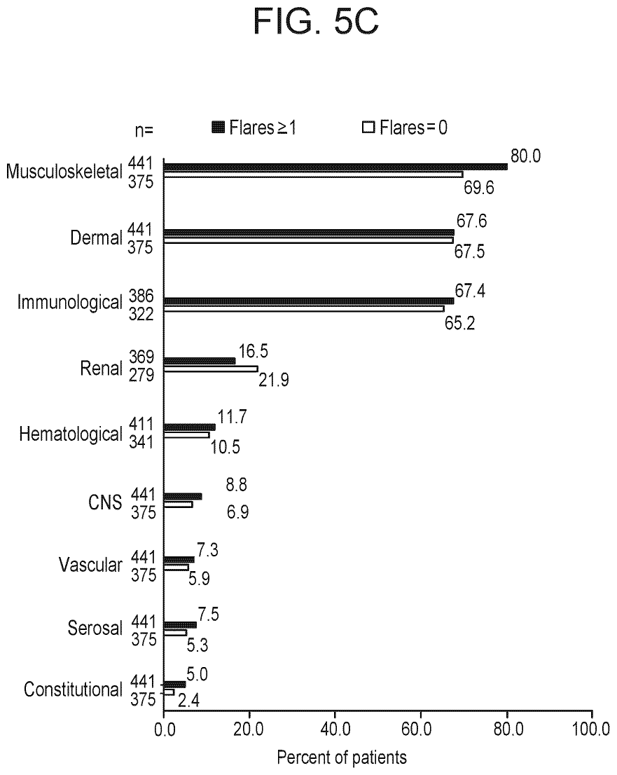

In the overall SPOCS cohort, patients most often had musculoskeletal (75.3%), dermal (67.6%), or immunological (66.4%) system involvement at baseline (FIG. 5A). The mean (SD) SLEDAI-2K score was 9.8 (4.6) and median (interquartile range [IQR]: 25th-75th percentile) score was 8.0 (6.0-12.0) (Table 7).

TABLE 7
Clinical characteristics of the SPOCS population at baseline, by SLEDAI-2K and IFNGS category

| | | SLEDAI-2K | | | IFNGS | | |
|---|---|---|---|---|---|---|---|
| | Overall | <10 | ≥10 | Nominal | High | Low | Nominal |
| | (N = 823) | (n = 343) | (n = 241) | P-value | (n = 522) | (n = 217) | P-value |
| Years since diagnosis | n = 814 | n = 340 | n = 238 | 0.012 | n = 517 | n = 215 | 0.052 |
| of SLE | | | | | | | |
| Mean ± SD | 11.1 ± 9.2 | 12.4 ± 9.6 | 10.4 ± 8.6 | | 11.7 ± 9.6 | 10.2 ± 8.6 | |
| Median (IQR) | 9.0 (4.0, | 10.0 (5.0, | 8.0 (3.0, | | 9.0 (4.0, | 8.0 (3.0, | |
| | 17.0) | 19.0) | 17.0) | | 18.0) | 16.0) | |
| Age at SLE first | n = 732 | n = 309 | n = 223 | 0.522 | n = 473 | n = 186 | <0.001 |
| manifestation, years | | | | | | | |
| Mean ± SD | 32.0 ± 13.9 | 31.6 ± | 30.8 ± 13.4 | | 30.0 # 12.7 | 36.8 ± 14.6 | |
| | | 13.9 | | | | | |
| Median (IQR) | 30.0 (22.0, | 30.0 | 29.0 (22.0, | | 28.0 (21.0, | 37.5 (25.5, | |
| | 41.0) | (20.2, | 37.0) | | 37.0) | 47.0) | |
| | | 42.0) | | | | | |
| Age at SLE first | n = 732 | n = 309 | n = 223 | 0.548 | n = 473 | n = 186 | <0.0001 |
| manifestation, years, n | | | | | | | |
| (%) | | | | | | | |
| <18 | 105 (14.3) | 52 (16.8) | 30 (13.5) | | 73 (15.4) | 17 (9.1) | |
| 18-29 | 245 (33.5) | 101 | 83 (37.2) | | 181 (38.3) | 43 (23.1) | |
| | | (32.7) | | | | | |
| 30-49 | 297 (40.6) | 123 | 90 (40.4) | | 182 (38.5) | 89 (47.9) | |
| | | (39.8) | | | | | |
| 50+ | 85 (11.6) | 33 (10.7) | 20 (9.0) | | 37 (7.8) | 37 (19.9) | |
| SLE diagnosis age, | n = 815 | n = 341 | n = 238 | 0.297 | n = 517 | n = 216 | <0.001 |
| years, n (%) | | | | | | | |
| <18 | 114 (14.0) | 57 (16.7) | 32 (13.5) | | 81 (15.7) | 18 (8.3) | |
| 18-29 | 231 (28.3) | 91 (26.7) | 77 (32.4) | | 180 (34.8) | 31 (14.4) | |
| 30-49 | 350 (42.9) | 144 (42.2) | 103 (43.3) | | 206 (39.8) | 112 (51.9) | |
| 50+ | 120 (14.7) | 49 (14.4) | 26 (10.9) | | 50 (9.7) | 55 (25.5) | |
| SLEDAI-2K | n = 584 | n = 343 | n = 241 | <0.001 | n = 386 | n = 135 | 0.101 |
| Mean ± SD | 9.8 ± 4.6 | 6.8 ± 1.3 | 13.9 ± 4.5 | | 9.8 ± 4.3 | 9.1 ± 4.9 | |
| Median (IQR) | 8.0 (6.0, | 7.0 (6.0, | 12.0 (10.0, | | 8.0 (6.0, | 8.0 (6.0, | |
| | 12.0) | 8.0) | 16.0) | | 12.0) | 10.0) | |
| SLEDAI-2K ≥10, n (%) | 241 (41.3) | 0 (0.0) | 241 | <0.001 | 164 (42.5) | 45 (33.3) | 0.062 |
| | | | (100.0) | | | | |
| Modified SLEDAI-2K^{a} | n = 817 | n = 343 | n = 241 | <0.001 | n = 519 | n = 217 | 0.174 |
| Mean ± SD | 6.5 ± 3.4 | 4.8 ± 1.9 | 8.5 ± 4.2 | | 6.2 ± 3.2 | 6.6 ± 3.4 | |
| Median (IQR) | 6.0 (4.0, 8.0) | 4.0 (4.0, | 8.0 (6.0, | | 6.0 (4.0, | 6.0 (4.0, | |
| | | 6.0) | 10.0) | | 8.0) | 8.0) | |
| Physician Global | n = 820 | n = 340 | n = 241 | <0.001 | n = 520 | n = 217 | 0.127 |
| Assessment | | | | | | | |
| Mean ± SD | 1.5 ± 0.6) | 1.4 (0.6) | 1.7 (0.6) | | 1.5 (0.6) | 1.5 (0.6) | |
| Median (IQR) | 1.5 (1.1, 2.0) | 1.4 (1.0, | 1.9 (1.3, | | 1.5 (1.1, | 1.5 (1.0, | |
| | | 2.0) | 2.0) | | 2.0) | 2.0) | |
| SDI total score | n = 811 | n = 336 | n = 237 | 0.100 | n = 512 | n = 217 | 0.181 |
| Mean ± SD | 1.2 ± 1.6 | 1.1 ± 1.4 | 1.3 ± 1.7 | | 1.1 ± 1.6 | 1.3 ± 1.6 | |
| Median (IQR) | 1.0 (0.0, 2.0) | 0.5 (0.0, | 1.0 (0.0, | | 0.0 (0.0, | 1.0 (0.0, | |
| | | 2.0) | 2.0) | | 2.0) | 2.0) | |
| SDI group, n (%) | | | | 0.599 | | | 0.135 |
| 0 | 386 (47.6) | 168 (50.0) | 109 (46.0) | | 258 (50.4) | 92 (42.4) | |
| 1 | 190 (23.4) | 70 (20.8) | 56 (23.6) | | 116 (22.7) | 55 (25.3) | |
| 2+ | 235 (29.0) | 98 (29.2) | 72 (30.4) | | 138 (27.0) | 70 (32.3) | |
| Any organ damage^{b}, n | 425 (52.4) | 168 (50.0) | 128 (54.0) | 0.344 | 254 (49.6) | 125 (57.6) | 0.048 |
| (%) | | | | | | | |
| Annualized flare ratec | n = 822 | n = 343 | n = 241 | 0.005 | n = 522 | n = 216 | 0.666 |
| Mean ± SD | 1.5 ± 2.2 | 1.3 ± 2.0 | 1.9 ± 2.4 | | 1.5 ± 2.2 | 1.6 ± 2.4 | |
| Median (IQR) | 2.0 (0.0, 2.0) | 2.0 (0.0, | 2.0 (0.0, | | 2.0 (0.0, | 2.0 (0.0, | |
| | | 2.1) | 2.0) | | 2.0) | 2.0) | |
| ≥1 flare^{d}, n (%) | 445 (54.1) | 175 (51.0) | 155 (64.3) | 0.001 | 287 (55.0) | 113 (52.3) | 0.508 |
| ≥1 mild flare^{d}, n (%) | 187 (22.8) | 95 (27.7) | 43 (17.8) | 0.006 | 124 (23.8) | 44 (20.4) | 0.318 |
| ≥1 moderate flare^{d}, n | 155 (18.9) | 62 (18.1) | 59 (24.5) | 0.060 | 93 (17.8) | 42 (19.4) | 0.603 |
| (%) | | | | | | | |
| ≥1 severe flare^{d}, n (%) | 64 (7.8) | 18 (5.3) | 31 (12.9) | 0.001 | 35 (6.7) | 22 (10.2) | 0.107 |
| Conditions or | n = 822 | n = 342 | n = 241 | | n = 522 | n = 217 | |
| comorbidities, n (%) | | | | | | | |
| Any comorbidity | 687/823 | 273/343 | 205 (85.1) | 0.091 | 426 (81.6) | 192 (88.5) | 0.022 |
| | (83.5) | (79.6) | | | | | |
| Joint disease (SLE/non- | 514 (62.5) | 201 (58.8) | 159 (66.0) | 0.078 | 306 (58.6) | 157 (72.4) | <0.001 |
| SLE) | | | | | | | |
| CVD/stroke risk factors | 276 (33.6) | 102 (29.8) | 74 (30.7) | 0.820 | 160 (30.7) | 92 (42.4) | 0.002 |
| Cancer | 47 (5.7) | 23 (6.7) | 12 (5.0) | 0.382 | 27 (5.2) | 15 (6.9) | 0.352 |
| Renal disease (ESRD) | 96 (11.7) | 43 (12.6) | 30 (12.5) | 0.964 | 74 (14.2) | 14 (6.5) | 0.003 |
| CNS disorders | 86 (10.5) | 24 (7.0) | 34 (14.1) | 0.005 | 51 (9.8) | 26 (12.0) | 0.370 |
| Diabetes mellitus (all) | 63 (7.7) | 31 (9.1) | 14 (5.8) | 0.147 | 32 (6.1) | 28 (12.9) | 0.002 |
| Metabolic syndrome | 20 (2.4) | 8 (2.3) | 6 (2.5) | 0.907 | 6 (1.1) | 13 (6.0) | <0.001 |
| Family history of | 224/805 | 91/327 | 72/239 | 0.551 | 148/510 | 59/213 | 0.720 |
| autoimmune disease | (27.7) | (27.8) | (30.1) | | (29.0) | (27.7) | |

All P-values are nominal; there have been no adjustments for multiplicity. Subgroups do not total overall population n due to missing data.
ªModified SLEDAI-2K score was defined as the SLEDAI-2K assessment score without the inclusion of points attributable to any urine or laboratory results including immunologic measures and lupus headache.
^{b}Systemic Lupus International Collaborating Clinics/American College of Rheumatology Damage Index >0.
^{c}The annualized flare rate is calculated by dividing the number of flares by the follow-up time in days and multiplying by 365.25.
^{d}Within 6 months of study entry.
ACR, American College of Rheumatology
SLE classification criteria;
CNS, central nervous system;
CVD, cardiovascular disease;
ESRD, end stage renal disease;
IFNGS, type I interferon gene signature;
IQR, interquartile range; PGA, Physician Global Assessment;
SD, standard deviation;
SDI, SLICC/ACR damage index;
SLE, systemic lupus erythematosus;
SLEDAI-2K, SLE Disease Activity Index 2000;
SLICC, Systemic Lupus International Collaborating Clinics;
SPOCS, SLE Prospective Observational Cohort Study.

The percentage of patients with baseline SLEDAI-2K organ domain involvement was higher among patients with baseline SLEDAI-2K scores >10, vs patients with baseline scores <10, for CNS (18.7% vs 0.9%), renal (32.0% vs 7.3%), immunological (83.0% vs 56.6%), and dermal (74.3% vs 58.3%) domains (FIG. 5B).

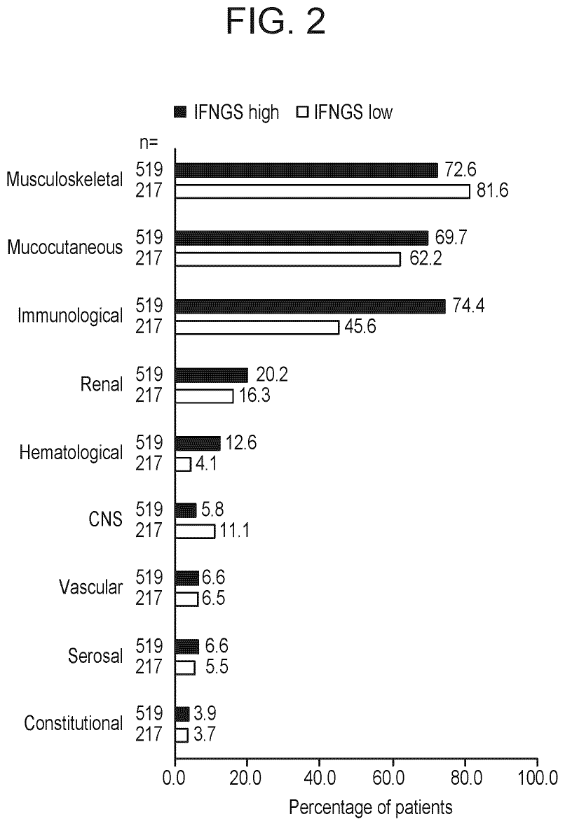

On average, modified SLEDAI-2K scores, SLEDAI-2K scores, and the percentage of patients with SLEDAI-2K scores >10 did not differ between IFNGS high and IFNGS low patients. More patients in the IFNGS-high group had hematological (12.6% vs 4.1%), immunological (74.4% vs 45.6%), or dermal (69.7% vs 62.2%) SLEDAI-2K domain involvement than in the IFNGS-low group (all P<0.05) (FIG. 2). Conversely, fewer IFNGS high patients had CNS (5.8% vs 11.1%; P=0.012) and musculoskeletal (72.6% vs 81.6%; P=0.010) involvement than patients in the IFNGS-low group. Renal domain involvement among patients in the IFNGS-high group was 20.2% versus 16.3% in the IFNGS-low group.

The mean (SD) SDI score was 1.2 (1.6), and approximately half of all patients had organ damage (52.4%, 425/811) (Table 7). A slightly higher percentage of patients in the IFNGS-low group had organ damage than in the IFNGS-high group (57.6% vs 49.6%; P=0.048).

### 6.3.3. Flares

Approximately half of all patients (54.1%, 445/822) had experienced >1 flare within the 6 months prior to the baseline visit (Table 7); organ system involvement was generally similar between those who had and had not experienced flare (FIG. 5C). Compared with patients with baseline SLEDAI-2K scores <10, those with SLEDAI-2K scores >10 had higher mean [SD] annualized flare rate in the year before enrollment (1.9 [2.4] vs 1.3 [2.0], P=0.005) and severe flares (according to physicians' assessments) were more common (12.9% vs 5.3%; P=0.001). In contrast, flares did not vary according to IFNGS status; neither the annualized flare rates or flare severities (all P=NS), nor the percentages of patients who experienced ≥1 flare in the 6 months prior to baseline visit (IFNGS high vs low: 55.0%, 287/522 vs 52.3%, 113/216; P=0.51) differed between the IFNGS high and low groups.

### 6.3.4. SLE Treatment

At baseline, 80.4% (662/823) of the patients were receiving antimalarial agents (53.8% (443/823) were receiving immunosuppressants, and 20.2% (166/823) were receiving biologics (Table 3). Oral glucocorticoids were in use or used in the past year by 61.5% (506/823) of patients. Among the patients receiving oral glucocorticoids, mean cumulative and daily doses were similar regardless of SLEDAI-2K or IFNGS group.

There were no differences in any other baseline treatments between the baseline SLEDAI-2K groups (all P≥0.5) (Table 8). More patients categorized as IFNGS high vs low received immunosuppressants (58.0% [303/522] vs 43.8% [95/217]; P<0.001) and oral glucocorticoids (67.8% [354/522] vs 49.3% [107/217]; P<0.001), whereas fewer patients in the IFNGS high vs low group received antimalarials (78.0% [407/522] vs 87.1% [189/217]; P=0.004). Higher percentages of patients in the IFNGS-high vs low group were receiving immunosuppressants, regardless of concurrent antimalarial use (Table 9).

TABLE 8
SLE medications in the SPOCS population at baseline, by SLEDAI-2K and IFNGS category

| | | SLEDAI-2K | | | IFNGS | | |
|---|---|---|---|---|---|---|---|
| | Overall | <10 | ≥10 | Nominal | High | Low | Nominal |
| | (N = 823) | (n = 343) | (n = 241) | P-value | (n = 522) | (n = 217) | P-value |
| Number of | n = 823 | n = 343 | n = 241 | 0.777 | n = 522 | n = 217 | <0.001 |
| medication classes | | | | | | | |
| (±AM) | | | | | | | |
| Mean ± SD | 1.3 ± 0.8 | 1.3 ± 0.8 | 1.3 ± 0.8 | | 1.4 ± 0.8 | 1.1 ± 0.8 | |
| Median (IQR) | 1.0 (1.0, | 1.0 (1.0, | 1.0 (1.0, | | 1.0 (1.0, | 1.0 (0.0, | |
| | 2.0) | 2.0) | 2.0) | | 2.0) | 2.0) | |
| Level of therapy, n | | | | 0.920 | | | <0.001 |
| (%) | | | | | | | |
| None (±AM) | 139 | 61 (17.8) | 38 (15.8) | | 71 (13.6) | 55 (25.3) | |
| | (16.9) | | | | | | |
| Monotherapy | 323 | 131 | 97 (40.3) | | 196 | 93 (42.9) | |
| (±AM) | (39.3) | (38.2) | | | (37.5) | | |
| Dual (±AM) | 313 | 131 | 92 (38.2) | | 218 | 61 (28.1) | |
| | (38.0) | (38.2) | | | (41.8) | | |
| Triple (±AM) | 48 (5.8) | 20 (5.8) | 14 (5.8) | | 37 (7.1) | 8 (3.7) | |
| Antimalarials, n (%) | 662 | 277 | 193 | 0.839 | 407 | 189 | 0.004 |
| | (80.4) | (80.8) | (80.1) | | (78.0) | (87.1) | |
| Immuno | 443 | 187 | 137 | 0.577 | 303 | 95 (43.8) | <0.001 |
| suppressants^{a}, n (%) | (53.8) | (54.5) | (56.9) | | (58.0) | | |
| Biologics^{b}, n (%) | 166 | 64 (18.7) | 40 (16.6) | 0.522 | 99 (19.0) | 45 (20.7) | 0.580 |
| | (20.2) | | | | | | |
| Glucocorticoids^{c} | 506 | 211 | 155 | 0.491 | 354 | 107 | <0.001 |
| | (61.5%) | (61.5%) | (64.3%) | | (67.8%) | (49.3%) | |
| Cumulative | n = 491 | n = 204 | n = 148 | 0.613 | n = 344 | n = 104 | 0.665 |
| glucocorticoids^{d}, g | | | | | | | |
| Mean ± SD | 2.5 ± 3.1 | 2.7 ± 3.2 | 2.5 ± 3.0 | | 2.5 ± 3.1 | 2.3 ± 2.9 | |
| Total | | | | 0.613 | | | 0.665 |
| glucocorticoids^{e}, | | | | | | | |
| mg/day | | | | | | | |
| Mean # SD | 6.8 ± 8.5 | 7.3 ± 8.9 | 6.8 ± 8.2 | | 6.8 ± 8.5 | 6.4 ± 7.8 | |
| Cumulative | n = 808 | n = 336 | n = 234 | 0.863 | n = 512 | n = 214 | 0.014 |
| glucocorticoids^{d}, g | | | | | | | |
| [including 0 mg] | | | | | | | |
| Mean ± SD | 1.5 ± 2.7 | 1.6 ± 2.8 | 1.6 ± 2.7 | | 1.7 ± 2.8 | 1.1 ± 2.3 | |
| Total | | | | 0.863 | | | 0.014 |
| glucocorticoids^{e}, | | | | | | | |
| mg/day [including 0 | | | | | | | |
| mgl | | | | | | | |
| Mean ± SD | 4.1 ± 7.4 | 4.4 ± 7.8 | 4.3 ± 7.3 | | 4.6 ± 7.7 | 3.1 = 6.3 | |
| Total glucocorticoids | | | | 0.829 | | | <0.001 |
| daily dose^{e}, n (%) | | | | | | | |
| No glucocorticoids | 317 | 132 | 86 (36.8) | | 168 | 110 | |
| | (39.2) | (39.3) | | | (32.8) | (51.4) | |
| Dose ≤7.5 mg | 353 | 142 | 103 | | 248 | 73 (34.1) | |
| | (43.7) | (42.3) | (44.0) | | (48.4) | | |
| Dose >7.5 mg | 138 | 62 (18.5) | 45 (19.2) | | 96 (18.8) | 31 (14.5) | |
| | (17.1) | | | | | | |

All P-values are nominal; there have been no adjustments for multiplicity. Subgroups do not total overall population n due to missing data.
^{a}Cytotoxic or immunosuppressive agents included cyclophosphamide, methotrexate, mycophenolate mofetil/mycophenolic acid (MMF/MPA), azathioprine, tacrolimus, mizoribine, leflunomide, or cyclosporine;
^{b}Belimumab or rituximab;
^{c}Number of patients with at least one ongoing glucocorticoid prescription or at least one prescription recorded in the previous year;
^{d}The sum of all glucocorticoids received in the past 365 days, standardized to g/year;
^{e}Cumulative glucocorticoids divided by 365 to convert to daily dose and group patients into daily dose groups.
AM, antimalarial;
Biologics, belimumab or rituximab;
IFNGS, type I interferon gene signature;
IQR, interquartile range;
SD, standard deviation;
SLE, systemic lupus erythematosus;
SLEDAI-2K, SLE Disease Activity Index 2000;
SLICC, Systemic Lupus International Collaborating Clinics;
SPOCS, SLE Prospective Observational Cohort Study.

TABLE 9
Baseline immunosuppressant and antimalarial use, by IFNGS status

| | | IFNGS | | |
|---|---|---|---|---|
| | Overall | High | Low | Nominal |
| n (%) | (N = 823) | (n = 522) | (n = 217) | P-value |
| Antimalarials | 662 (80.4) | 407 (78.0) | 189 (87.1) | 0.004 |
| Antimalarials without | 313 (38.0) | 172 (33.0) | 109 (50.2) | <0.001 |
| immunosuppressants^{a} | | | | |
| Antimalarials with | 349 (42.4) | 235 (45.0) | 80 (36.9) | 0.041 |
| immunosuppressants | | | | |
| Immunosuppressants | 443 (53.8) | 303 (58.0) | 95 (43.8) | <0.001 |
| Immunosuppressants without | 94 (11.2) | 68 (13.0) | 15 (6.9) | 0.0165 |
| antimalarials | | | | |
| Immunosuppressants with | 349 (42.4) | 235 (45.0) | 80 (36.9) | 0.041 |
| antimalarials | | | | |

^{a}Cytotoxic or immunosuppressive agents included cyclophosphamide, methotrexate, mycophenolate mofetil/mycophenolic acid, azathioprine, tacrolimus, mizoribine, leflunomide, or cyclosporine.
IFNGS, type I interferon gene signature.

### 6.3.5. Characteristics by Country

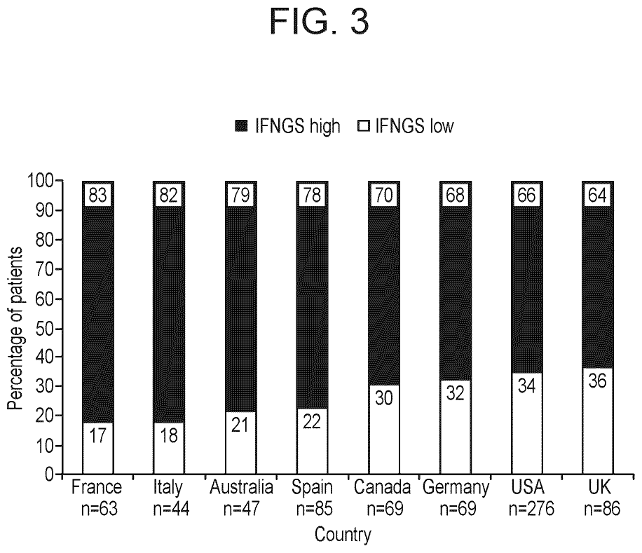

The percentage of IFNGS high patients ranged from 64% to 83% across countries (FIG. 3). Overall baseline data by country are provided in the online supplement (Table 10-Table 12).

TABLE 10
Baseline demographics and patient characteristics of the SPOCS population by country

| | Australia | Canada | USA | France | Germany | Italy | Spain | UK | Nominal |
|---|---|---|---|---|---|---|---|---|---|
| | (n = 51) | (n = 81) | (n = 308) | (n = 64) | (n = 74) | (n = 52) | (n = 97) | (n = 96) | P-value |
| Age, years | n = 51 | n = 81 | n = 308 | n = 64 | n = 74 | n = 52 | n = 97 | n = 96 | 0.1062 |
| Mean ± SD | 43.7 ± | 46.1 ± | 45.9 ± | 42.8 ± | 47.8 ± | 41.3 ± | 45.4 ± | 45.1 ± | |
| | 13.6 | 15.2 | 14.9 | 13.5 | 12.5 | 13.3 | 12.0 | 12.7 | |
| Median (IQR) | 45.0 | 45.0 | 45.0 | 42.0 | 50.5 | 42.0 | 44.0 | 45.0 | |
| | (34.0, | (33.0, | (33.0, | (32.0, | (36.0, | (30.5, | (38.0, | (35.0, | |
| | 52.0) | 57.0) | 56.5) | 52.0) | 56.0) | 49.0) | 53.0 | 54.5 | |
| Age >40 | 29 (56.9) | 47 | 194 | 36 | 48 (64.9) | 30 | 63 | 56 | 0.845 |
| years, n (%) | | (58.0) | (63.0) | (56.2) | | (57.7) | (64.9) | (58.3) | |
| Male, n (%) | 7 (13.7) | 6 (7.4) | 14 (4.6) | 7 (10.9) | 9 (12.2) | 3 (5.8) | 8 (8.2) | 2 (2.1) | 0.0331 |
| Race, n (%) | n = 50 | n = 81 | n = 293 | n = 41 | n = 74 | n = 51 | n = 96 | n = 93 | <0.001 |
| Asian | 16 (32.0) | 21 | 20 (6.8) | 0 (0.0) | 2 (2.7) | 1 (2.0) | 0 (0.0) | 11 | |
| | | (25.9) | | | | | | (11.8) | |
| Black | 1 (2.0) | 6 (7.4) | 92 | 11 | 2 (2.7) | 0 (0.0) | 2 (2.1) | 14 | |
| | | | (31.4) | (26.8) | | | | (15.1) | |
| White | 29 (58.0) | 44 | 168 | 29 | 70 (96.4) | 49 | 90 | 62 | |
| | | (54.3) | (57.3) | (70.7) | | (96.1) | (93.8) | (66.7) | |
| Other | 4 (8.0) | 10 | 13 (4.4) | 1 (2.4) | 0 (0.0) | 1 (2.0) | 4 (4.2) | 6 (6.5) | |
| | | (12.3) | | | | | | | |
| Ethnicity, n | n = 1 | n = 81 | n = 304 | n = 25 | n = 72 | n = 52 | n = 96 | n = 91 | <0.001 |
| (%) | | | | | | | | | |
| Hispanic or | 0 (0) | 3 (3.7) | 49 | 3 (12.0) | 1 (1.4) | 6 | 49 | 2 (2.2) | |
| Latino | | | (16.1) | | | (11.5) | (51.0) | | |
| BMI kg/m² | n = 31 | n = 74 | n = 283 | n = 63 | n = 73 | n = 47 | n = 87 | n = 69 | <0.001 |
| Mean ± SD | 28.7 ± | 25.9 ± | 29.3 ± | 24.8 ± | 26.0 ± | 23.3 ± | 25.0 ± | 27.4 ± | |
| | 5.3 | 5.5 | 7.5 | 6.3 | 5.2 | 4.8 | 5.5 | 6.6 | |
| Median (IQR) | 28.4 | 24.9 | 27.8 | 22.7 | 25.3 | 21.8 | 24.0 | 26.5 | |
| | (24.9, | (21.9, | (24.2, | (20.0, | (22.0, | (19.9, | (21.0, | (22.0, | |
| | 32.1) | 28.0) | 32.9) | 28.4) | 28.3) | 26.0) | 27.3) | 31.6) | |
| BMI category, | n = 31 | n = 74 | n = 283 | n = 63 | n = 73 | n = 47 | n = 87 | n = 69 | <0.001 |
| n (%) | | | | | | | | | |
| Underweight | 1 (3.2) | 3 (4.1) | 9 (3.2) | 8 (12.7) | 3 (4.1) | 4.(8.5) | .3 (3.4) | 2 (2.9) | |
| Normal | 7 (22.6) | 34 | 77 | 29 | 31 (42.5) | 29 | 50 | 27 | |
| | | (45.9) | (27.2) | (46.0) | | (61.7) | (57.5) | (39.1) | |
| Overweight | 9 (29.0) | 24 | 90 | 15 | 27 (37.0) | 10 | 19 | 18 | |
| | | (32.4) | (31.8) | (23.8) | | (21.3) | (21.8) | (26.1) | |
| Obese | 14 (45.2) | 13 | 107 | 11 | 12 (16.4) | 4 (8.5) | 15 | 22 | |
| | | (17.6) | (37.8) | (17.5) | | | (17.2) | (31.9) | |

All P-values are nominal; there have been no adjustments for multiplicity. Subgroups do not total overall
population n due to missing data.
BMI, body mass index;
IQR, interquartile range;
SD, standard deviation;
SLE, systemic lupus erythematosus;
SPOCS, SLE Prospective Observational Cohort Study;
UK, United Kingdom;
U.S.A., United States of America.

TABLE 11
Baseline demographics and patient characteristics of the SPOCS population by country

| | Australia | Canada | USA | France | Germany | Italy | Spain | UK | Nominal |
|---|---|---|---|---|---|---|---|---|---|
| | (n = 51) | (n = 81) | (n = 308) | (n = 64) | (n = 74) | (n = 52) | (n = 97) | (n = 96) | P-value |
| Years since | n = 51 | n = 81 | n = 300 | n = 63 | n = 74 | n = 52 | n = 97 | n = 96 | 0.337 |
| diagnosis of SLE | | | | | | | | | |
| Mean ± SD | 13.0 ± | 11.5 ± | 11.0 ± | 8.7 ± | 11.4 ± | 10.2 ± | 11.9 ± | 11.4 ± | |
| | 10.5 | 9.8 | 9.3) | 7.3 | 8.0 | 9.1 | 10.0 | 8.8 | |
| Median (IQR) | 12.0 | 10.0 | 8.0 | 7.0 | 10.5 | 8.0 | 9.0 | 10.0 | |
| | (5.0, | (3.0, | (4.0, | (1.5, | (5.0, | (1.8, | (3.0, | (3.5, | |
| | 18.0) | 18.0) | 17.0) | 15.5) | 15.8) | 17.2) | 18.0) | 19.0) | |
| Age at first SLE | n = 50 | n = 73 | n = 283 | n = 63 | n = 40 | n = 49 | n = 86 | n = 88 | 0.554 |
| manifestation | | | | | | | | | |
| Mean ± SD | 29.0 ± | 33.2 ± | 32.6 ± | 31.2 ± | 34.1 ± | 30.0 ± | 32.4 ± | 31.1 ± | |
| | 13.1 | 13.5 | 14.3 | 13.6 | 14.3 | 14.3 | 13.3 | 13.4 | |
| Median (IQR) | 26.5 | 31.0 | 30.0 | 30.0 | 32.0 | 27.0 | 32.0 | 28.0 | |
| | (18.2, | (26.0, | (22.0, | (23.0, | (22.8, | (20.0, | (22.2, | (21.0, | |
| | 37.8) | 41.0) | 43.0) | 39.0) | 41.8) | 38.0) | 41.8) | 41.0) | |
| SLE diagnosis age | n = 51 | n = 81 | n = 300 | n = 64 | n = 74 | n = 52 | n = 97 | n = 96 | 0.041 |
| (years), n (%) | | | | | | | | | |
| <18 | 9 (17.6) | 15 | 38 | 11 | 3 (4.1) | 9 | 13 | 16 | |
| | | (18.5) | (12.7) | (17.2) | | (17.3) | (13.4) | (16.7) | |
| 18-29 | 20 (39.2) | 11 | 86 | 16 | 25 (33.8) | 21 | 26 | 26 | |
| | | (13.6) | (28.7) | (25.0) | | (40.4) | (26.8) | (27.1) | |
| 30-49 | 16 (31.4) | 43 | 129 | 27 | 31 (41.9) | 15 | 50 | 39 | |
| | | (53.1) | (43.0) | (42.2) | | (28.8) | (51.5) | (40.6) | |
| 50+ | 6 (11.8) | 12 | 47 | 10 | 15 (20.3) | 7 | 8 (8.2) | 15 | |
| | | (14.8) | (15.7) | (15.6) | | (13.5) | | (15.6) | |
| SLEDAI-2K | n = 49 | n = 72 | n = 192 | n = 54 | n = 41 | n = 39 | n = 79 | n = 58 | <0.001 |
| Mean ± SD | 8.4 ± 3.4 | 11.0 ± | 8.7 ± | 9.8 ± | 10.6 ± | 11.1 ± | 10.6 ± | 10.1 ± | |
| | | 5.6 | 3.7 | 4.8 | 4.6 | 5.0 | 5.5 | 4.5 | |
| Median (IQR) | 8.0 | 9.0 | 8.0 | 8.0 | 10.0 | 10.0 | 8.0 | 8.0 | |
| | (6.0, 9.0) | (7.0, | (6.0, | (6.0, | (8.0, | (8.0, | (7.0, | (8.0, | |
| | | 13.2) | 10.0) | 12.0) | 12.0) | 12.0) | 12.0) | 12.0) | |
| SLEDAI-2K ≥10, n | 12 (24.5) | 35 | 68 | 22 | 22 (53.7) | 21 | 36 | 25 | 0.031 |
| (%) | | (48.6) | (35.2) | (40.7) | | (53.8) | (45.6) | (43.1) | |
| Modified SLEDAI- | n = 50 | n = 81 | n = 308 | n = 63 | n = 74 | n = 51 | n = 96 | n = 94 | 0.016 |
| 2K^{a} | | | | | | | | | |
| Mean ± SD | 4.9 ± 3.3 | 6.6 ± | 6.5 ± | 6.1 ± | 6.6 ± 3.9 | 5.9 ± | 6.6 ± | 7.2 ± | |
| | | 3.6 | 2.9 | 3.1 | | 3.4 | 4.4 | 3.4 | |
| Median (IQR) | 4.0 | 6.0 | 6.0 | 4.0 | 6.0 | 4.0 | 6.0 | 6.0 | |
| | (2.0, 6.0) | (4.0, | (4.0, | (4.0, | (4.0, 8.0) | 4.0, | (4.0, | (4.0, | |
| | | 8.0) | 8.0) | 8.0) | | 8.0) | 8.0) | 8.0) | |
| Physician Global | n = 50 | n = 81 | n = 306 | n = 64 | n = 74 | n = 52 | n = 97 | n = 96 | <0.001 |
| Assessment | | | | | | | | | |
| Mean ± SD | 1.1 ± 0.6 | 1.3 ± | 1.5 ± | 1.5 ± | 1.7 ± 0.5 | 1.6 ± | 1.8 ± | 1.7 ± | |
| | | 0.6 | 0.5 | 0.7 | | 0.5 | 0.5 | 0.5 | |
| Median (IQR) | | 1.3 | 1.5 | 1.5 | | 1.5 | 2.0 | 1.8 | |
| | 1.0 | (1.0, | (1.0, | (1.0, | 1.9 | (1.0, | 1.5 | (1.3, | |
| | (0.9, 1.5) | 1.7) | 2.0) | 2.0) | (1.3, 2.0) | 2.0) | 2.0) | 2.0) | |
| SDI total score | n = 39 | n = 81 | n = 308 | n = 64 | n = 74 | n = 52 | n = 97 | n = 96 | 0.021 |
| Mean ± SD | 1.1 ± 1.5 | 1.3 ± | 1.4 ± | 0.6 ± | 1.1 ± 1.6 | 0.8 ± | 1.1 ± | 1.2 ± | |
| | | 1.7 | 1.6 | 1.1 | | 1.2 | 1.7 | 1.7 | |
| Median (IQR) | 0.0 | 1.0 | 1.0 | 0.0 | 0.0 | 0.0 | 0.0 | 1.0 | |
| | (0.0, 2.0) | (0.0, | (0.0, | (0.0, | (0.0, 1.8) | (0.0, | (0.0, | (0.0, | |
| | | 2.0) | 2.0) | 1.0) | | 1.0) | 2.0) | 2.0) | |
| SDI group, n (%) | n = 39 | n = 81 | n = 308 | n = 64 | n = 74 | n = 52 | n = 97 | n = 96 | 0.012 |
| 0 | 21 (53.8) | 36 | 118 | 42 | 40 (54.1) | 30 | 52 | 47 | |
| | | (44.4) | (38.3) | (65.6) | | (57.7) | (53.6) | (49.0) | |
| 1 | 7 (17.9) | 18 | 85 | 13 | 15 (20.3) | 13 | 19 | 20 | |
| | | (22.2) | (27.6) | (20.3) | | (25.0) | (19.6) | (20.8) | |
| 2+ | 11 (28.2) | 27 | 105 | 9 (14.1) | 19 (25.7) | 9 | 26 | 29 | |
| | | (33.3) | (34.1) | | | (17.3) | (26.8) | (30.2) | |
| Any organ damage, | 18 (46.2) | 45 | 190 | 22 | 34 (45.9) | 22 | 45 | 49 | <0.001 |
| n (%) | | (55.6) | (61.7) | (34.4) | | (42.3) | (46.4) | (51.0) | |
| Annualized flare | n = 51 | n = 81 | n = 307 | n = 64 | n = 74 | n = 52 | n = 97 | n = 96 | <0.001 |
| rateb | | | | | | | | | |
| Mean ± SD | 0.7 ± 1.5 | 1.5 ± | 1.4 ± | 1.7 ± | 0.8 ± 1.9 | 1.8 ± | 2.3 ± | 1.8 ± | |
| | | 1.2 | 2.3 | 1.2 | | 1.0 | 2.9) | 3.0 | |
| Median (IQR) | 0.0 | 2.0 | 0.0 | 2.0 | 0.0 | 2.0 | 2.0 | 2.0 | |
| | (0.0, 0.0) | (0.0, | (0.0, | (2.0, | (0.0, 1.5) | (2.0, | (0.0, | (0.0, | |
| | | 2.0) | 2.0) | 2.0) | | 2.0) | 2.0) | 2.0) | |
| ≥1 flare^{c}, n (%) | 12 (23.5) | 54 | 149 | 50 | 19 (25.7) | 44 | 59 | 58 | <0.001 |
| | | (66.7) | (48.5) | (78.1) | | (84.6) | (60.8) | (60.4) | |
| ≥1 mild flare^{c}, n (%) | 3 (5.9) | 14 | 83 | 19 | 7 (9.5) | 24 | 22 | 15 | <0.001 |
| | | (17.3) | (27.0) | (29.7) | | (46.2) | (22.7) | (15.6) | |
| ≥1 moderate flare^{c}, | 0 (0.0) | 25 | 51 | 22 | 1 (1.4) | 8 | 17 | 31 | <0.001 |
| n (%) | | (30.9) | (16.6) | (34.4) | | (15.4) | (17.5) | (32.3) | |
| ≥1 severe flare^{c}, n | 1 (2.0) | 11 | 17 (5.5) | 8 | 10 (13.5) | 4 (7.7) | 5 (5.2) | 8 (8.3) | 0.040 |
| (%) | | (13.6) | | (12.5%) | | | | | |
| Conditions and | n = 51 | n = 81 | n = 308 | n = 64 | n = 74 | n = 52 | n = 97 | n = 96 | |
| comorbidities, n | | | | | | | | | |
| (%) | | | | | | | | | |
| Any comorbidity | 30 (58.8) | 74 | 266 | 52 | 64 (86.5) | 46 | 75 | 80 | <0.001 |
| | | (91.4) | (86.4) | (81.2) | | (88.5) | (77.3) | (83.3) | |
| Joint disease | 3 (5.9) | 64 | 212 | 43 | 42 (56.8) | 39 | 44 | 67 | <0.001 |
| (SLE/non-SLE) | | (79.0) | (69.1) | (67.2) | | (75.0) | (45.4) | (69.8) | |
| CVD/stroke risk | 11 (21.6) | 28 | 127 | 13 | 31 (41.9) | 9 | 29 | 28 | <0.001 |
| factors | | (34.6) | (41.4) | (20.3) | | (17.3) | (29.9) | (29.2) | |
| Cancer | 2 (3.9) | 5 (6.2) | 25 (8.1) | 2 (3.1) | 4 (5.4) | 1 (1.9) | 5 (5.2) | 3 (3.1) | 0.414 |
| Renal disease | 17 (33.3) | 13 | 31 | 8 (12.5) | 9 (12.2) | 5 (9.6) | 6 (6.2) | 7 (7.3) | <0.001 |
| (ESRD) | | (16.0) | (10.1) | | | | | | |
| CNS disorders | 7 (13.7) | 10 | 24 (7.8) | 4 (6.2) | 6 (8.1) | 6 | 16 | 13 | 0.210 |
| | | (12.3) | | | | (11.5) | (16.5) | (13.5) | |
| Diabetes mellitus | 1 (2.0) | 4 (4.9) | 35 | 7 (10.9) | 6 (8.1) | 3 (5.8) | 2 (2.1) | 5 (5.2) | 0.029 |
| (all) | | | (11.4) | | | | | | |
| Metabolic syndrome | 0 (0.0) | 3 (3.7) | 7 (2.3) | 1 (1.6) | 3 (4.1) | 3 (5.8) | 0 (0.0) | 3 (3.1) | 0.339 |
| Family history of | 10/34 | 31 | 97 | 15 | 16 (21.6) | 15 | 12 | 28 | 0.005 |
| autoimmune disease | (29.4) | (38.3) | (31.6) | (23.4) | | (28.8) | (12.4) | (29.2) | |

All P-values are nominal; there have been no adjustments for multiplicity. Subgroups do not total overall population n due to missing data.
^{a}Modified SLEDAI-2K score defined as the SLEDAI-2K assessment score without the inclusion of points attributable to any urine or laboratory results including immunologic measures and lupus headache.
^{b}The annualized flare rate is calculated by dividing the number of flares by the follow-up time in days and multiplying by 365.25.
^{c}Within 6 months of study entry.
ACR, American College of Rheumatology
SLE classification criteria;
CVD, cardiovascular disease;
ESRD, end stage renal disease;
IQR, interquartile range;
SD, standard deviation;
SDI, SLICC/ACR damage index;
SLE, systemic lupus erythematosus;
SLEDAI-2K, Systemic Lupus Erythematosus Disease Activity Index 2000;
SLICC, Systemic Lupus International Collaborating Clinics;
SPOCS, SLE Prospective Observational Cohort Study;
UK, United Kingdom;
USA, United States of America.

TABLE 12
Clinical characteristics of the SPOCS population at baseline, by country

| | Australia | Canada | USA | France | Germany | Italy | Spain | UK | Nominal |
|---|---|---|---|---|---|---|---|---|---|
| | (n = 51) | (n = 81) | (n = 308) | (n = 64) | (n = 74) | (n = 52) | (n = 97) | (n = 96) | P-value |
| Number of medication | n = 51 | n = 81 | n = 308 | n = 64 | n = 74 | n = 52 | n = 97 | n = 96 | <0.001 |
| classes (±AM) | | | | | | | | | |
| Mean ± SD | 1.6 ± 0.8 | 1.3 ± 0.7 | 1.2 ± 0.8 | 1.4 ± 0.8 | 1.6 ± 0.8 | 1.8 ± 0.7 | 1.4 ± 0.9 | 1.2 ± 0.9 | |
| Median (IQR) | 2.0 (1.0, | 1.0 (1.0, | 1.0 (1.0, | 2.0 (1.0, | 2.0 (1.0, | 2.0 (1.0, | 2.0 (1.0, | 1.0 (0.0, | |
| | 2.0) | 2.0) | 2.0) | 2.0) | 2.0) | 2.0) | 2.0) | 2.0) | |
| Level of therapy, n | n = 51 | n = 81 | n = 308 | n = 64 | n = 74 | n = 52 | n = 97 | n = 96 | <0.001 |
| (%) | | | | | | | | | |
| None (±AM) | 6 (11.8) | 12 | 62 | 10 | 3 (4.1) | 1 | 18 | 27 | |
| | | (14.8) | (20.1) | (15.6) | | (1.9) | (18.6) | (28.1) | |
| Monotherapy (±AM) | 12 (23.5) | 35 | 145 | 20 | 31 (41.9) | 17 | 30 | 33 | |
| | | (43.2) | (47.1) | (31.2) | | (32.7) | (30.9) | (34.4) | |
| Dual (±AM) | 29 (56.9) | 32 | 91 | 32 | 30 (40.5) | 28 | 41 | 30 | |
| | | (39.5) | (29.6) | (50.0) | | (53.8) | (42.3) | (31.2) | |
| Triple (±AM) | 4 (7.8) | 2 (2.5) | 10 | 2 (3.1) | 10 (13.5) | 6 | 8 | 6 | |
| | | | (3.3) | | | (11.5) | (8.2) | (6.2) | |
| Antimalarials, n (%) | 43 (84.3) | 66 | 249 | 54 | 55 (74.3) | 39 | 82 | 74 | 0.580 |
| | | (81.5) | (80.8) | (84.4) | | (75.0) | (84.5) | (77.1) | |
| Immunosuppressants^{a}, | 39 (76.5) | 53 | 142 | 27 | 49 (66.2) | 31 | 49 | 53 | <0.001 |
| n (%) | | (65.4) | (46.1) | (42.2) | | (59.6) | (50.5) | (55.2) | |
| Biologics^{b}, n (%) | 6 (11.8) | 4 (4.9) | 79 | 14 | 16 (21.6) | 21 | 17 | 9 | <0.001 |
| | | | (25.7) | (21.9) | | (40.4) | (17.5) | (9.4) | |
| OCS^{c}, n (%) | 37 (72.6) | 55 | 145 | 50 | 57 (77.0) | 41 | 70 | 51 | <0.001 |
| | | (67.9) | (47.1) | (78.1) | | (78.8) | (72.2) | (53.1) | |
| Cumulative | n = 32 | n = 51 | n = 142 | n = 48 | n = 56 | n = 41 | n = 70 | n = 51 | <0.001 |
| glucocorticoids^{d}, g | | | | | | | | | |
| Mean ± SD | 5.7 ± 6.3 | 2.4 ± 3.1 | 2.2 ± 2.9 | 2.0 ± 1.7 | 2.0 ± 2.5 | 3.0 ± 3.2 | 2.1 ± 1.7 | 2.5 ± 2.4 | |
| Total glucocorticoids^{e}, | | | | | | | | | <0.001 |
| mg/day | | | | | | | | | |
| Mean ± SD | 15.5 ± | 6.6 ± | 6.2 ± | 5.4 ± | 5.4 ± 6.9 | 8.2 ± | 5.7 ± | 6.7 ± | |
| | 17.3 | 8.5 | 8.0 | 4.5 | | 8.7 | 4.7 | 6.6 | |
| Cumulative | n = 46 | n = 77 | n = 305 | n = 62 | n = 73 | n = 52 | n = 97 | n = 96 | <0.001 |
| glucocorticoids^{d}, g | | | | | | | | | |
| [including 0 mg] | | | | | | | | | |
| Mean ± SD | 3.9 ± 5.9 | 1.6 ± 2.8 | 1.0 ± 2.3 | 1.5 ± 1.7 | 1.5 ± 2.4 | 2.3 ± 3.1 | 1.5 ± 1.7 | 1.3 ± 2.1 | |
| Total glucocorticoids^{e}, | n = 46 | n = 77 | n = 305 | n = 62 | n = 73 | n = 52 | n = 97 | n = 96 | |
| mg/day [including | | | | | | | | | |
| 0 mg] | | | | | | | | | |
| Mean ± SD | 10.8 ± | 4.4 ± | 2.8 ± | 4.2 ± | 4.2 ± 6.4 | 6.4 ± | 4.1 ± | 3.6 ± | |
| | 16.1 | 7.6 | 6.3 | 4.6 | | 8.4 | 4.7 | 5.9 | |
| Total glucocorticoid | n = 46 | n = 77 | n = 305 | n = 62 | n = 73 | n = 52 | n = 97 | n = 96 | <0.001 |
| daily dose^{e}, n (%) | | | | | | | | | |
| No glucocorticoids | 14 (30.4) | 26 | 163 | 14 | 17 (23.3) | 11 | 27 | 45 | |
| | | (33.8) | (53.4) | (22.6) | | (21.2) | (27.8) | (46.9) | |
| Dose ≤7.5 mg | 16 (34.8) | 37 | 102 | 33 | 48 (65.8) | 28 | 54 | 35 | |
| | | (48.1) | (33.4) | (53.2) | | (53.8) | (55.7) | (36.5) | |
| Dose >7.5 mg | 16 (34.8) | 14 | 40 | 15 | 8 (11.0) | 13 | 16 | 16 | |
| | | (18.2) | (13.1) | (24.2) | | (25.0) | (16.5) | (16.7) | |

All P-values are nominal; there have been no adjustments for multiplicity. Subgroups do not total overall population n due to missing data.
^{a}Cytotoxic or immunosuppressive agents included cyclophosphamide, methotrexate, mycophenolate mofetil/mycophenolic acid (MMF/MPA), azathioprine, tacrolimus, mizoribine, leflunomide, or cyclosporine;
^{b}Belimumab or rituximab;
^{c}Number of patients with at least one ongoing glucocorticoid prescription or at least one prescription recorded in the previous year;
^{d}The sum of all glucocorticoids received in the past 365 days, standardized to g/year;
^{e}Cumulative glucocorticoids divided by 365 to convert to daily dose and group patients into daily dose groups.
AM, antimalarial; Biologics, belimumab or rituximab;
IFNGS, type I interferon gene signature;
IQR, inter-quartile range;
SD, standard deviation;
SLE, systemic lupus erythematosus;
SLEDAI-2K, SLE Disease
Activity Index 2000;
SLICC, Systemic Lupus International Collaborating Clinics;
SPOCS, SLE Prospective Observational Cohort Study.

### 6.4. Discussion

SPOCS is a prospective, observational study designed to systematically describe the patient journey in terms of clinical outcomes over time of a large, real-world cohort of patients with moderate to severe SLE. Overall, SPOCS identified substantial clinical burden in a broadly representative group of patients with moderate to severe SLE in a real-world setting; SPOCS patients had a mean SLE duration of 11 years, approximately half of them had experienced at least 1 flare within the 6 months before study enrollment, and more than half had evidence of organ damage at baseline. Additionally, the inventors characterized patients with high disease activity and those with elevated IFN signaling according to IFNGS-high status to gain insight into two subgroups of patients with higher medical need.

Patients with higher SLE disease activity tend to experience greater clinical, social, and economic burdens than those with low disease activity. In the SPOCS SLE cohort, approximately 41% of patients had high disease activity at baseline, based on SLEDAI-2K scores ≥10. Patients with SLEDAI-2K≥10 were younger and had shorter SLE duration than those with SLEDAI-2K<10. At baseline, the percentages of patients in the SLEDAI-2K≥10 group with organ-specific manifestations in the immunological, mucocutaneous, renal, vascular, hematologic, and serosal systems were notably higher compared with the SLEDAI<10 group. SPOCS patients with higher baseline SLEDAI-2K scores (≥10) experienced more flares and more severe flares than patients with lower baseline scores, consistent with other real-world studies. There were no differences in therapy between the baseline SLEDAI-2K groups, which will aid the examination of the impact of treatment in these two groups longitudinally in this study.

SPOCS, the first real-world study to evaluate patients' IFNGS levels, found that baseline IFNGS status was associated with differences in patient characteristics. Patients in the IFNGS-high group were younger at SLE first manifestation than those in the IFNGS-low group, with the percentage of patients in the IFNGS-high group under 30 years old at SLE diagnosis being twice that of the IFNGS-low group. Conversely, the IFNGS-low group had twice the proportion of patients with “late-onset” SLE (ie, patients diagnosed with SLE at ≥50 years old). Age of onset, here identified as “age of SLE diagnosis”, has been associated with differences in clinical outcomes and disease progression. For example, late-onset SLE less frequently presents with organ-specific manifestations and tends to be more indolent. This is consistent with the patterns observed in this cohort, as IFNGS-high patients were more likely to have hematological, immunological, and dermal involvement than IFNGS-low patients. We identified less musculoskeletal involvement in IFNGS-high vs -low patients in our real-world study; similar findings were identified in IFNGS-high vs -low patients with moderate to severe SLE in a clinical trial setting. Slightly more IFNGS-low vs -high patients had organ damage according to SDI in our study, which is likely a function of the older age of the IFNGS-low patients, as evidenced by their more frequent comorbidities. Possibly owing to differences in disease activity and manifestations at baseline, more patients in the IFNGS-high group than in the low group received immunosuppressants and/or oral glucocorticoids.

Notably, the relative proportions of patients who were IFNGS-high vs IFNGS-low were higher among Black and Asian patients compared with White patients, which supports findings in other moderate to severe SLE populations. Three of the 4 genes included in the SPOCS IFNGS assay (IFI27, IFI44L, and RSAD2) have been associated with African ancestry; in the same meta-analysis (which adjusted for the presence of LN and HCQ use), only IFI27 expression was associated with disease activity.

SPOCS is a prospective study designed to systematically describe the patient journey in clinical practice for patients enrolled from multiple centers across a range of countries. The study was designed to broadly represent the moderate to severe SLE population. In contrast to many SLE-related real-world studies that rely on existing electronic health records in insurance claims databases, this study started with the collection of very detailed baseline data, which was then collected longitudinally according to the same procedures.

### REFERENCES

All publications mentioned in the specification are herein incorporated by reference in their entirety.
[1]E. Lever, M. R. Alves, and D. A. Isenberg, Pharmacogenomics Pers. Med. 13, 39 (2020).
[2]G. J. Pons-Estel et al., Semin. Arthritis Rheum. 39, 257 (2010).
[3]D. P. McCauliffe, Semin. Cutan. Med. Surg. 20, 14 (2001).
[4]L. Uva et al., Autoimmune Dis. 2012, 834291 (2012).
[5]A. S. Zayat et al., Rheumatol. Oxf. Engl. 55, 485 (2016).
[6]C. Sa et al., Rheumatology (Oxford, England) 48, (2009).
[7]I. B. Murimi-Worstell et al., BMJ Open 10, e031850 (2020).
[8]C. LINDHOLM et al., WO2022223714A1 (27 Oct. 2022).
[9]M. Khamashta et al., Ann. Rheum. Dis. 75, 1909 (2016).
[10] R. Tummala et al., Lupus Sci. Med. 5, e000252 (2018).
[11] B. W. Higgs et al., Ann. Rheum. Dis. 73, 256 (2014).
[12] R. Furie et al., Arthritis Rheumatol. Hoboken Nj 69, 376 (2017).
[13] R. A. Furie et al., Lancet Rheumatol. 1, e208 (2019).
[14] E. F. Morand et al., N. Engl. J. Med. 382, 211 (2020).
[15] Y. Tanaka and R. Tummala, Mod. Rheumatol. 0, 1 (2020).
[16] G. T. Ferguson et al., J. Asthma Allergy 11, 63 (2018).
[17] Z. Touma, M. Urowitz, and D. Gladman, Lupus 19, 49 (2010).
[18] L. A. Criswell, Bull. NYU Hosp. Jt. Dis. 66, 176 (2008).
[19] S. Sigurdsson et al., Hum. Mol. Genet. 17, 872 (2008).
[20] S. Sigurdsson et al., Hum. Mol. Genet. 17, 2868 (2008).
[21] A. A. Bengtsson et al., Lupus 9, 664 (2000).
[22] M. C. Dall'era et al., Ann. Rheum. Dis. 64, 1692 (2005).
[23] E. C. Baechler et al., Proc. Natl. Acad. Sci. U.S.A 100, 2610 (2003).
[24] L. Bennett et al., J. Exp. Med. 197, 711 (2003).
[25] M. K. Crow and J. Wohlgemuth, Arthritis Res. Ther. 5, 279 (2003).
[26] X. Feng et al., Arthritis Rheum. 54, 2951 (2006).
[27] K. A. Kirou et al., Arthritis Rheum. 50, 3958 (2004).
[28] K. A. Kirou et al., Arthritis Rheum. 52, 1491 (2005).
[29] Y. Yao et al., Hum. Genomics Proteomics HGP 2009, 374312 (2009).
[30] X. Huang et al., Cell. Signal. 20, 112 (2008).
[31] W. Hylton et al., Ann. Rheum. Dis. 45, 220 (1986).
[32] H. Okamoto et al., Arthritis Rheum. 50, 3731 (2004).
[33] Y. Tanaka and R. Tummala, Mod. Rheumatol. 31, 1 (2021).
[34] ACR Meeting Abstracts (n.d.).
[35] E. R. Hammond et al., BMJ Open 10, e036563 (2020).
[36] P. K. Brohawn et al., Ann. Rheum. Dis. 77, 1704 (2018).
[37] L. Chiche et al., Arthritis Rheumatol. 66, 1583 (2014).
[38] W. P. Kennedy et al., Lupus Sci. Med. 2, e000080 (2015).
[39] E. F. Morand et al., N. Engl. J. Med. 382, 211 (2020).

1. A method of treating systemic lupus erythematosus (SLE) in a subject in need thereof, comprising administering a therapeutically effective amount of an inhibitor of type I IFN signalling to the subject, wherein:
a) the subject was diagnosed with SLE at an age of less than (<) 50 years, and/or
b) the subject experienced their first SLE manifestation at an age of about 30 years or less.

2. The method of claim 1, wherein the subject was diagnosed with SLE at an age of <30 years.

3. The method of claim 1, wherein the subject was diagnosed with SLE at an age of 18 to 29.

4. The method of claim 1, wherein the subject was diagnosed with SLE at an age of <18 years.

5. The method of claim 1, wherein the inhibitor of type I IFN signalling is a human monoclonal antibody specific for IFNAR1.

6. The method of claim 5, wherein the antibody comprises:
(a) a heavy chain variable region complementarity determining region 1 (HCDR1) comprising the amino acid sequence of SEQ ID NO: 3;
(b) a heavy chain variable region complementarity determining region 2 (HCDR2) comprising the amino acid sequence of SEQ ID NO: 4;
c) a heavy chain variable region complementarity determining region 3 (HCDR3) comprising the amino acid sequence of SEQ ID NO: 5;
(d) a light chain variable region complementarity determining region 1 (LCDR1) comprising the amino acid sequence SEQ ID NO: 6;
(e) a light chain variable region complementarity determining region 2 (LCDR2) comprising the amino acid sequence SEQ ID NO: 7; and
(f) a light chain variable region complementarity determining region 3 (LCDR3) comprising the amino acid sequence SEQ ID NO: 8.

7. The method of claim 5, wherein the antibody comprises: (a) a human heavy chain variable region comprising the amino acid sequence of SEQ ID NO: 1; and (b) a human light chain variable region comprising the amino acid sequence of SEQ ID NO: 2.

8. The method of claim 5, wherein the antibody comprises in the Fc region an amino acid substitution of L234F, as numbered by the EU index as set forth in Kabat and wherein said antibody exhibits reduced affinity for at least one Fc ligand compared to an unmodified antibody.

9. The method of claim 5, wherein the antibody comprises: (a) a human heavy chain comprising the amino acid sequence of SEQ ID NO: 11; and (b) a human light chain comprising the amino acid sequence of SEQ ID NO: 12.

10. The method of claim 5, wherein the IFNAR1 inhibitor is anifrolumab or a functional variant thereof.

11. The method of claim 10, comprising administering an intravenous dose of anifrolumab or the functional variant thereof to the subject.

12. The method of claim 11, wherein the intravenous dose is ≥300 mg anifrolumab or the functional variant thereof.

13. The method of claim 10, wherein the intravenous dose is ≤1000 mg.

14. The method of claim 13, wherein the intravenous dose is about 300 mg, about 900 mg or about 1000 mg.

15. The method of claim 11, wherein the intravenous dose is administered every four weeks (Q4W)

16. The method of claim 10, comprising administering a subcutaneous dose of anifrolumab or the functional variant thereof.

17. The method of claim 16, wherein the subcutaneous dose is >105 mg and <150 mg anifrolumab or the functional variant thereof.

18. The method of claim 16, wherein the subcutaneous dose is <135 mg anifrolumab or the functional variant thereof.

19. The method of claim 16, wherein the subcutaneous dose is about 120 mg.

20. The method of claim 16, wherein the subcutaneous dose is administered in a single administration step.

21. The method of claim 16, wherein the subcutaneous dose is administered at intervals of 6-8 days.

22. The method of claim 16, wherein the subcutaneous dose is administered once per week.

23. The method of claim 16, wherein the subcutaneous dose has a volume of about 0.5 to about 1 ml.

24. The method of claim 23, wherein the subcutaneous dose has a volume of about 0.8 ml.

25. The method of claim 1, wherein the subject is a type I interferon stimulated gene signature (IFNGS)-test high patient pre-treatment.

26. The method of claim 1, comprising identifying the subject as IFNGS-test high patient pre-treatment.

27. A pharmaceutical composition for use in the method of claim 1, wherein the pharmaceutical composition comprises anifrolumab or a functional variant thereof.

28. An injection device comprising the pharmaceutical composition of claim 27.

29. The injection device of claim 28, wherein the injection device is a pre-filled syringe (PFS).

30. The injection device of claim 28, wherein the injection device is an accessorized pre-filed syringe (AFPS).

31. The injection device of claim 28, wherein the injection device is an auto-injector.

32. A kit comprising the injection device of claim 28, and instructions for use.

33. The kit of claim 32, wherein the instructions for use comprise the instructions for use specify that the subject a subject that was diagnosed with SLE at an age of less than (<) 50 years, and/or that a subject that experienced their first SLE manifestation at an age of about 30 years or less.


SEQUENCE LISTING (24 sequences)

SEQ ID NO: 1 - protein, 117 aa, synthetic construct
EVQLVQSGAEVKKPGESLKISCKGSGYIFTNYWIAWVRQMPGKGLESMGLLYPGDSDIRYSPSFQGQVTISADKSITTAYLQWSSLKASDTAMYYCARHDIEGFDYWGRGTLVTVSS

SEQ ID NO: 2 - protein, 108 aa, synthetic construct [sequence omitted]

SEQ ID NO: 3 - protein, 5 aa, synthetic construct
NYWIA

SEQ ID NO: 4 - protein, 17 aa, synthetic construct
IIYPGDSDIRYSPSFQG

SEQ ID NO: 5 - protein, 8 aa, synthetic construct
HDIEGFDY

SEQ ID NO: 6 - protein, 12 aa, synthetic construct
RASQSVSSSFFA

SEQ ID NO: 7 - protein, 7 aa, synthetic construct
GASSRAT

SEQ ID NO: 8 - protein, 9 aa, synthetic construct
QQYDSSAIT

SEQ ID NO: 9 - protein, 107 aa, synthetic construct [sequence omitted]

SEQ ID NO: 10 - protein, 330 aa, synthetic construct
  source 1..330: note = Heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 447 aa, synthetic construct
  source 1..447: note = Heavy chain
EVQLVQSGAEVKKPGESLKISCKGSGYIFTNYWIAWVRQMPGKGLESMGIIYPGDSDIRYSPSFQGQVTISADKSITTAYLQWSSLKASDTAMYYCARHDIEGFDYWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 215 aa, synthetic construct
  source 1..215: note = Light chain
EIVLTQSPGTLSLSPGERATLSCRASQSVSSSFFAWYQQKPGQAPRLLIYGASSRATGIPDRLSGSGSGTDFTLTITRLEPEDFAVYYCQQYDSSAITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 80 aa, synthetic construct
EVQLVQSGAEVKKPGESLRISCKGSGYTFTNYWVAWVRQMPGKGLESMGIIYPGDSDTRYSPSFQGHVTISADKSISTAY

SEQ ID NO: 14 - protein, 108 aa, synthetic construct
DIQMTQSPSSLSASLGDRVTITCRASQNVGNYLNWYQQKPGKAPKLLIYRASNLASGVPSRFSGSGSGTDFTLTISSLQPEDFATYYCQQMEHAPPTFGQGTKVEIKR

SEQ ID NO: 15 - protein, 108 aa, synthetic construct
EIVLTQSPGTLSLSPGERATLSCRASQSVIGYYLAWYQQKPGQAPRLLIYSVSTLASGIPDRFSGSGSGTDFTLTISRLEPEDFAVYYCQQYYRFPITFGQGTKVEIK

SEQ ID NO: 16 - protein, 117 aa, synthetic construct
EVQLVQSGAEVKKPGESLRISCKGSGYTFTNYWMAWVRQMPGKGLESMGIIYPSDSDTRYSPSFQGHVTISADKSISTAYLQWSSLKASDTAMYYCARHDVEGYDYWGQGTLVTVSS

SEQ ID NO: 17 - protein, 119 aa, synthetic construct
EVQLVESGGGLVQPGGSLRLSCAASGFSLSSYYMTWVRQAPGKGLEWVSVINVYGGTYYASWAKGRFTISRDNSKNTLYLQMNSLRAEDTAVYYCAREDVAVYMAIDLWGQGTLVTVSS

SEQ ID NO: 18 - protein, 111 aa, synthetic construct
AIQMTQSPSSLSASVGDRVTITCQASQSISNQLSWYQQKPGKAPKLLIYDASSLASGVPSRFSGSRSGTKFTLTISSLQPEDFATYYCLGIYGDGADDGIAFGGGTKVEIK

SEQ ID NO: 19 - protein, 5 aa, synthetic construct
SYYMT

SEQ ID NO: 20 - protein, 16 aa, synthetic construct
VINVYGGTYYASWAKG

SEQ ID NO: 21 - protein, 11 aa, synthetic construct
EDVAVYMAIDL

SEQ ID NO: 22 - protein, 11 aa, synthetic construct
QASQSISNQLS

SEQ ID NO: 23 - protein, 7 aa, synthetic construct
DASSLAS

SEQ ID NO: 24 - protein, 13 aa, synthetic construct
LGIYGDGADDGIA